(19) United States
(12) Patent Application Publication
(10) Pub. No.: US 2026/0355162 A1
(43) Pub. Date: Nov. 12, 2026

(54) INHIBITION OF ENDOPLASMIC RETICULUM OXIDOREDUCTIN 1 MODULATES NEURONAL EXCITABILITY AND NOCICEPTIVE SENSITIVITY

(71) Applicants: The Governors of the University of Alberta, Edmonton (CA); Board of Regents, The University of Texas System, Austin, TX (US)

(72) Inventors: Bradley James Kerr, Edmonton (CA); Thomas Simmen, Edmonton (CA); Aislinn Dawn Maguire, Edmonton (CA); Muhammad Saad Yousuf, Edmonton (CA)

(21) Appl. No.: 19/671,717

(22) Filed: May 8, 2026

Related U.S. Application Data
(60) Provisional application No. 63/802,883, filed on May 9, 2025.

(51) Int. Cl. A61K 31/4155; A61K 31/343; A61P 29/00

(52) U.S. Cl. CPC A61K 31/4155; A61K 31/343; A61P 29/00

(57) ABSTRACT

There is described a method of treating pain in a subject in need thereof, comprising: administering a therapeutically effected amount an inhibitor of Endoplasmic Reticulum Oxidoreductin 1 (EOR1).

### CROSS REFERENCE TO RELATED APPLCATIONS

This application claims priority to US provisional patent application number 63/802,883, filed May 9, 2025, the entire contents of which is hereby incorporated by reference.

### STATEMENT REGARDING FEDERALLY SPONSORED RESEARCH OR DEVELOPMENT

This invention was made with Federal Government support under contract R01 DK134893 awarded by the National Institutes of Health (NIH). The government has certain rights in the invention.

### FIELD

The present disclosure relates generally to inhibition of Endoplasmic Reticulum Oxidoreductin 1 (ERO 1) in the treatment of pain.

### BACKGROUND

### 1. Introduction

Neuronal hyperexcitability and spontaneous activity are hallmarks of acute and chronic pain conditions. Calcium has emerged as a major contributor to these phenomena due to its effects on gene expression, mitochondria, neurotransmitter release, ion channels, and action potential propagation.¹⁻³ Nociceptors in the peripheral nervous system are relatively easier targets than pain pathways in the central nervous system, making them attractive targets for pharmacological manipulation of pain signalling.⁴

The endoplasmic reticulum (ER) and mitochondria are the two largest repositories of calcium in the cell. These two organelles are also physically and functionally tethered by ER-mitochondria contact sites (ERMCS).^{6,7} One of many functions of ERMCS is to allow for calcium transfer from the ER to mitochondria.⁸ ER calcium is released by IP3Rs, which are tethered to mitochondria.^{6,9} This allows calcium to flow through the mitochondrial calcium uniporter (MCU) channel into the mitochondrial matrix.¹⁰ Increased cytosolic calcium leads to changes in cell excitability as noted above, but increased calcium transfer also regulates mitochondrial function, which is tied to excitability and pain.¹¹ Calcium has been shown to drive changes to the mitochondrial membrane potential, the TCA cycle, electron transport chain flux, and substrate availability, leading to increased ATP production.^{12,13} Not only is more ATP needed to support the high energy demand of hyperactive cells,¹¹ but ATP may also be released by these cells, acting as a transmitter that can sensitize other neurons in the pain pathway.¹⁴ Increased mitochondrial metabolic activity also leads to production of reactive oxygen species (ROS) and the oxidative stress induced by ROS can alter cell signalling and ion channel activity.^{15,16}

Calcium release from the ER is increased in pain conditions such as nerve injury,^{1,17} and high calcium uptake by mitochondria has also been linked to neuronal hyperexcitability.^{11,18} Indeed, calcium regulation between these organelles has become a therapeutic target in pain research. In the chronic pain field, mouse models of diabetic neuropathy and chemotherapy-induced neuropathy have been used to study ER and mitochondrial calcium dysregulation.^{1,19,20} For example, in a diabetic neuropathy model, selective MCU deletion from mitochondria in nociceptors reduced their hyperexcitability and ultimately, nociceptive behaviour.²¹ Here, we sought to study this pathway in acute models of pain-like behaviour, and rather than target mitochondria, we focused upstream on ER calcium release.

Endoplasmic reticulum oxidoreductin 1 (ERO1) is an enzyme known for its involvement in oxidative protein folding in the ER, producing ROS as a by-product.²² ERO1 also has a critical role within the ER at ERMCS, whereby its production of ROS creates an oxidizing environment that stabilizes inositol 1,4,5 trisphosphate receptors (IP3Rs) in an open conformation.²³ These open IP3Rs at ERMCS allow for more calcium transfer from the ER to mitochondria.

### SUMMARY

In some examples there is provided the following embodiments:
1. A method of treating pain in a subject in need thereof, comprising: administering a therapeutically effected amount an inhibitor of Endoplasmic Reticulum Oxidoreductin 1 (EOR1).
2. The method of embodiment 1, wherein the inhibitor of EOR1 comprises EN460 and/or T151742.
3. The method of embodiment 2, wherein the pain is acute pain, chronic pain, inflammatory pain, nociceptive pain, neuropathic pain, hyperalgesia, allodynia, central pain, cancer-related pain, post-operative pain, visceral pain, musculo-skeletal pain, heart or vascular pain, head pain including migraine, orofacial pain including dental pain, or back pain.
4. The method of embodiment 2, wherein the pain is nociceptive pain.
5. The method of embodiment 4, wherein the nociceptive pain is post-operative pain, cluster headache pain, dental pain, surgical pain, pain resulting from burns, pain resulting from sunburns, pain resulting from exposure to extremely cold temperatures, bruises, fractures, post-partum pain, angina pain, genitourinary tract related pain, and/or damage by contact with toxic of hazardous chemicals.
6. The method of embodiment 2, wherein the pain is inflammatory pain.
7. The method of embodiment 6, wherein the inflammatory pain is arthritic pain, pain resulting from osteoarthritis, pain resulting from rheumatoid arthritis, pain resulting from inflammatory bowel diseases, pain resulting from psoriasis, or pain resulting from eczema.
8. The method of embodiment 2, wherein the subject is a human.
9. A method of treating pain in a subject in need thereof, comprising:
   assessing the subject to determine if they are experiencing pain, and if such pain is diagnosed;
   administering to the patient a therapeutically effective amount of an inhibitor of Endoplasmic Reticulum Oxidoreductin 1 (EOR1).
10. The method of embodiment 9, wherein the inhibitor of EOR1 comprises EN460 and/or T151742.
11. The method of embodiment 10, wherein the pain is acute pain, chronic pain, inflammatory pain, nociceptive pain, neuropathic pain, hyperalgesia, allodynia, central pain, cancer-related pain, post-operative pain, visceral pain, musculo-skeletal pain, heart or vascular pain, head pain including migraine, orofacial pain including dental pain, or back pain.
12. The method of embodiment 10, wherein the pain is nociceptive pain.
13. The method of embodiment 12, wherein the nociceptive pain is post-operative pain, cluster headache pain, dental pain, surgical pain, pain resulting from burns, pain resulting from sunburns, pain resulting from exposure to extremely cold temperatures, bruises, fractures, post-partum pain, angina pain, genitourinary tract related pain, and/or damage by contact with toxic of hazardous chemicals.
14. The method of embodiment 10, wherein the pain is inflammatory pain.
15. The method of embodiment 14, wherein the inflammatory pain is arthritic pain, pain resulting from osteoarthritis, pain resulting from rheumatoid arthritis, pain resulting from inflammatory bowel diseases, pain resulting from psoriasis, or pain resulting from eczema.
16. The method of embodiment 10, wherein the subject is a human.

### BRIEF DESCRIPTION OF THE FIGURES

Embodiments of the present disclosure will now be described, by way of example only, with reference to the attached Figures.

FIG. 1. Mouse dorsal root ganglia neurons express ERO1α. (A-C) Representative images of ERO1α and CGRP co-staining. (D) Quantification of ERO1α-CGRP co-stained cells as a percentage of total CGRP-positive cells. (E-G) Representative images of ERO1 and P2X3 co-staining. (H) Quantification of ERO1α-P2X3 co-stained cells as a percentage of total P2X3-positive cells. (I-K) Representative images of ERO1α and NF200 co-staining. (L) Quantification of ERO1α-NF200 co-stained cells as a percentage of total NF200-positive cells. Bars represent mean ±SD. n=8 female and 10 male animals per group.

FIG. 2. ERO1 inhibition blocks acute thermal pain, formalin-induced inflammatory pain, and formalin-induced spinal dorsal horn neuronal activation. (A) Dose-response curve for EN460 in the thermal tail flick test in naïve mice. (B) Time spent performing nociceptive behaviour in 15-minute intervals (phase I and II) after hind paw formalin injection and 20-minute pre-treatment with either vehicle (DMSO) or 10 mg/kg EN460, n=6 animals per group. ****p<0.0001 Uncorrected Fisher’s LSD test, Repeated Measures Two-way ANOVA. (C) Time spent performing nociceptive behaviour in 15-minute intervals (phase I and II) after hind paw formalin injection and 20-minute pre-treatment with either vehicle (DMSO) or 25 mg/kg EN460, n=5-7 animals per group. ****p<0.0001 Uncorrected Fisher’s LSD test, Repeated Measures Two-way ANOVA. (E) Time spent on rotarod before falling, before injections and 35 minutes after formalin injection, n=6 animals per group. Sidak’s multiple comparisons test, Repeated Measures Two-way ANOVA. (F, G) Representative images of c-FOS staining in spinal dorsal horn in formalin-injected mice pre-treated with either vehicle or EN460. (H) Quantification of c-FOS-positive cells per mm2 in the spinal dorsal horn, n=6 animals per group. Bars represent mean ± SD. ****p <0.0001 Unpaired t test.

FIG. 3. ERO1 inhibition reduces mechanical post-surgical pain in both male and female mice. (A) Timeline of behaviour, surgery, and daily EN460 treatment. (B) Male and (C) Female mechanical pain measured by Von Frey testing, expressed as weight required to trigger paw withdrawal, n=5 animals per group. * p<0.05 Tukey’s multiple comparisons test, Repeated Measures Two-way ANOVA. (D) Male and (E) Female thermal pain measured by Hargreaves testing, expressed as stimulus time required to trigger paw withdrawal, n=5 animals per group. Data is expressed as mean ± SD. Tukey’s multiple comparisons test, Repeated Measures Two-way ANOVA.

FIG. 4. ERO1 inhibition reduces DRG neuron excitability in vitro. (A&B) Representative traces of male and female untreated vs. EN460-treated neurons with 3s, 40pA current injections. dotted line denotes 0 mV, and the resting membrane potential of the neuron is denoted at the start of the sweep with a 0 pA current injection. (C) Resting Membrane potential of male and female DRG neurons with or without 10 μM EN460 treatment, n=37-120 cells per group. *p<0.05 Sidak’s multiple comparisons test, Two-way ANOVA. (D) Rheobase of male and female DRG neurons either with or without 10 μM EN460 treatment. *p<0.05, ****p<0.0001 Sidak’s multiple comparisons test, Two-way ANOVA. (E) Example traces of control or EN460-treated single firing DRG neurons highlighting the minimal current injection required to generate the action potential (rheobase), n=35-63 cells per group. (F) Percentages of male and female untreated or EN460-treated DRG neurons displaying either non-firing, single firing, or repetitive firing action potentials in response to a range of current injections from 0 pA to 2 nA in 10 or 100 pA increments, n=17-31 cells per group. (G) Male and (H) Female DRG neuron action potential frequency in response to 10 mV increment increasing current injections (0-60 pA), n=17-31 cells per group. *p<0.05 Student’s t test. Data is expressed as mean ± SD.

FIG. 5. ERO1 inhibition reduces mitochondrial membrane potential in DRG cultures. (A) Acute and (B) Chronic timelines of EN460 treatment and live staining. (C&D) Traces of male and female TMRM staining intensity over time after acute treatment with EN460, n=8 wells per group across two experiments. (E) Representative 10x image of TMRM staining. (F) Acute EN460 treatment represented as fold change of AUC of TMRM intensity over 40 minutes, normalized to sex-respective untreated wells, n=8 wells per group across two experiments. *p<0.05, ****p<0.0001 Sidak’s multiple comparisons test, ###p <0.001 (treatment factor) Two-way ANOVA. (G) Chronic EN460 treatment represented as fold change of TMRM staining normalized to sex-respective untreated wells, n=16 wells per group across four experiments. #p<0.05 (treatment factor) Two-way ANOVA. Bars represent mean ± SD.

FIG. 6. Chronic ERO1 inhibition reduces mitochondrial ATP production in DRG cultures. (A) Timeline of EN460 treatment. Representative traces of (B) oxygen consumption rate and (C) extracellular acidification rate, n=3 wells per group from one experiment. (D) Total ATP production represented as fold change of pmol/min/cell normalized to normalized to sex-respective untreated wells, n=9 wells per group across three experiments. *p<0.05 Sidak’s multiple comparisons test, #p<0.05 (treatment factor) Two-way ANOVA. (E) Percentage of total ATP production due to either glycolytic or mitochondrial ATP in male and female DRG cultures either untreated or treated with 48 hours of 10 M EN460, n=9 wells per group across three experiments. (F) Glycolytic ATP production represented as fold change of pmol/min/cell normalized to normalized to sex-respective untreated wells, n=9 wells per group across three experiments. (G) Mitochondrial ATP production represented as fold change of pmol/min/cell normalized to sex-respective untreated wells, n=9 wells per group across three experiments. **p<0.01 Sidak’s multiple comparisons test, ##p<0.01 (treatment factor) Two-way ANOVA. Bars represent mean ± SD.

FIG. 7. ERO1 inhibition reduces mitochondrial ROS production and does not increase cell death in DRG cultures. (A) Timeline of EN460 treatment and live staining. (B-E) Representative images of MitoTracker CM-H2TMRos staining. (F) Fold change of percentage of NucBlue-positive cells co-stained with MitoTracker CM-H2TMRos normalized to sex-respective untreated wells, n=8 wells per group across two experiments. *p<0.05 Sidak’s multiple comparisons test, ##p<0.01 (treatment factor) Two-way ANOVA. (G) Fold change of percentage of NucBlue-positive cells co-stained with Propidium Iodide normalized to sex-respective untreated wells, n=12 wells per group across three experiments. Bars represent mean ± SD.

FIG. 8. ERO1 is expressed in human sensory neurons, and ERO1 inhibition reduces human DRG neuron excitability in vitro. (A-D) Representative images of DAPI, ERO1, and peripherin antibody staining in post-mortem human DRGs. (E) Correlation of DRG neuron size with ERO1 antibody stain intensity, n= 228 cells. (F-H) Single cell RNA sequencing analysis of ERO1α mRNA expression in human DRG neuron subtypes using previously published data 50,51. (I) Representative calcium fluorescence traces of vehicle and EN460-treated human DRGs after KCl depolarization in vitro. (J) Quantification of fluorescence amplitude, n=43-55 cells per group across three experiments. *p<0.05, Unpaired t test. Bars represent mean ± SD.

FIG. 9. Formalin experiment reveals an effect of EN460, with no sex differences. (A) two male and (B) three female animals per condition were injected with 10 mg/kg EN460 or vehicle control 30 minutes prior to formalin hindpaw injection. *p<0.05, **p<0.01, ****p<0.0001 Repeated Measures Two-way ANOVA, Tukey’s multiple comparisons.

FIG. 10. Conditioned place preference testing does not reveal an intrinsically rewarding effect of EN460. Animals were injected with either drug of saline, then 15 minutes later they were placed in boxes for 30 minutes of conditioning. Mice were conditioned twice per day with 4-5 hours in between rounds. (A) On day 6, animals were tested for a preference between the saline and drug-paired chambers. (B) On day 7 animals were given EN460 15 minutes before testing to assess state-dependent preference. Bars indicate % of time spent in the paired chamber upon testing. No statistical significance was observed with Two-way ANOVA.

FIG. 11. ERO inhibition with T151742 reduces DRG neuron excitability in vitro. (A) Resting membrane potential of male and female DRG neurons either with or without 5 µM T151742 treatment. ***p<0.0001 Sidak’s multiple comparison test, Two-way ANOVA. (B) Rheobase of male and female DRG neurons either with or without 5 µM T151742 treatment. ****p<0.0001 Sidak’s multiple comparison test, Two-way ANOVA. (C) Percentages of male and female DRG neurons untreated or T151742-treated which were either non-firing, single firing, or repetitive firing in response to a range of current injections from 0 pA to 2 nA in 10 or 100 pA increments. (D) Male and (E) Female ERG neuron action potential frequency in response to increasing current injection. *p<0.05, **p<0.01, ***p<0.0001 Student’s t test. N=12-72 cells per group. Data is expressed as mean ± SD.

FIG. 12. Acute ARO1 inhibition with T151742 reduces mitochondrial membrane potential in DRG cultures. (A) Male and (B) Female cells treated acutely after baseline measurement with 5 µM T151742 exhibit decreased TMRM strain fluorescent intensity at 1 and 5 minutes after drug treatment. Expressed as fold change of integrated fluorescence intensity normalized to respective baselines. *p<0.05, **p<0.01, ***p<0.0001 Sidak’s multiple comparisons, Two-way ANOVA.

FIG. 13. Mouse and human DRGs have limited ERO1β expression. (A/B) Images from naïve mouse DRGs stained with DAPI and antibody for ERO1β. (C/D) RNA sequencing analysis in human DRGs reveals fewer cells express ERO1β.

### DETAILED DESCRIPTION

In some examples, there is provided a method of treating pain and/or reducing pain in a subject in need thereof, comprising: administering a therapeutically effective amount of an inhibitor of Endoplasmic Reticulum Oxidoreductin 1 (ERO1) in a patient in need thereof.

In some examples, the Endoplasmic Reticulum Oxidoreductin 1 (ERO1) inhibitor is EN460 or T151742. In further examples, the ERO1 inhibitor is QM295 (CAS No. 1241046-32-1) or M6766 (696628-90-7).

The term “pain” includes but is not limited to acute pain; chronic pain; inflammatory pain; nociceptive pain; neuropathic pain; hyperalgesia; allodynia; central pain; cancer-related pain; post-operative pain; visceral pain; musculo-skeletal pain; heart or vascular pain; head pain including migraine; orofacial pain, including dental pain; and back pain.

In some examples, pain may be acute and/or spontaneous. In some examples pain may be chronic and/or on-going.

The term “nociceptive pain” as used herein refers to pain caused by activity in primary sensory pain fibers in the peripheral nervous system. Neurons in the peripheral nervous system that typically respond to noxious or painful stimuli are commonly referred to as nociceptors or nociceptive neurons. In some examples, the nociceptive pathways extend to the somatosensory cortex.

As used herein, the term “nociceptive pain” involves direct activation of the nociceptors, such as mechanical, chemical, and thermal receptors, found in various tissues, such as bone, muscle, vessels, viscera, and cutaneous and connective tissue. Nociceptive pain occurs in the setting of an undamaged nervous system, e.g. the afferent somatosensory pathways are considered intact.

Non-limiting examples of nociceptive pain include post-operative pain, cluster headaches, dental pain, surgical pain, pain resulting from burns, sunburns, exposure to extremely cold temperatures, bruises, fractures, post-partum pain, angina pain, genitourinary tract related pain, damage by contact with toxic of hazardous chemicals.

The term “inflammatory pain” includes pain associates with active inflammation that may be caused by, for example, trauma, surgery, infection and autoimmune diseases.

Non-limiting examples of “inflammatory pain” includes arthritic pain, pain resulting from osteoarthritis , pain resulting from rheumatoid arthritis, pain resulting from inflammatory bowel diseases, pain resulting from psoriasis, or pain resulting from eczema.

As used herein, the terms “treat”, “treating” and “treatment” include the eradication, removal, reversion, alleviation, modification, or control of pain after its onset.

As used herein, the terms “prevention”, “preventing”, “preventive”, “prevent” and “prophylaxis” refer to the capacity of a therapeutic to avoid, minimize or difficult the onset or development of a disease or condition before its onset, in this case pain.

As used herein, the term “reducing pain” refers to alleviating pain localized at a site of interest.

As used herein, “treating” or “treatment” and/or “preventing” or “prevention”, as a whole, is meant at least a suppression or an amelioration of the symptoms associated with the condition afflicting the subject, where suppression and amelioration are used in a broad sense to refer to at least a reduction in the magnitude of a parameter, e.g., symptom associated with the condition being treated, such as pain. As such, the method of the present invention also includes situations where the condition is completely inhibited, e.g., prevented from happening, or stopped, e.g., terminated, such that the subject no longer experiences the condition. As such, the present method includes both preventing and managing pain.

Accordingly, treatment of pain includes, but is not limited to, preventing, ameliorating, controlling, reducing incidence of, or delaying the development or progression of pain.

The term “subject”, as used herein, refers to any human or non-human animal whom would benefit from treatment. Non-limiting examples of a subject include humans, non-human mammal, primates, rodents, companion animals (including but not limited to dogs, cats, mice, rats), livestock (including but not limited to horses, sheep, cattle, pigs), reptiles, amphibians, and the like. In a specific example, the subject is a human.

The term “treatment”, “treat”, “treating”, or lessening of severity” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. "Treating" and "Treatment" can also mean prolonging survival as compared to expected survival if not receiving treatment.

The term "amelioration" or "ameliorates" as used herein refers to a decrease, reduction or elimination of a condition, disease, disorder, or phenotype, including an abnormality or symptom.

In a specific example, treatment is carried out in vivo.

In a specific example, treatment is carried out in vitro, including but not limited to, in test tube, in cultured cells (both adherent cells and non-adherent cells), and the like.

In a specific example, treatment is carried out ex vivo, including but not limited to, in test tube, in cultured cells (both adherent cells and non-adherent cells), and the like.

The terms “inhibition”, “inhibiting”, “inhibit,” or “inhibitor” refer to the ability of a compound to reduce, slow, halt or prevent activity of a particular biological process in a cell relative to vehicle.

The term “pharmaceutically effective amount” as used herein refers to the amount of a drug or pharmaceutical agent that will elicit the biological or medical response of a tissue, system, animal or human that is being sought by a researcher or clinician. This amount can be a therapeutically effective amount.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert. The terms “physiologically tolerable carriers” and “biocompatible delivery vehicles” are used interchangeably. Thus, the term “carrier” or “excipient” may refer to a non-toxic solid, semi-solid or liquid filler, diluent. The term includes solvents, dispersion, media, coatings, isotonic agents, and adsorption delaying agents, and the like. The use of such media and agents for pharmaceutically active substances is well known in the art.

As used herein, the term “pharmaceutically-acceptable salts” refers to the conventional nontoxic salts or quaternary ammonium salt. These salts can be prepared in situ in the administration vehicle or the dosage form manufacturing process, or by separately reacting a compound in its free base or acid form with a suitable organic or inorganic acid or base, and isolating the salt thus formed during subsequent purification. Conventional nontoxic salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

A “pharmaceutical composition” as used herein refers to a chemical or biological composition suitable for administration to a mammalian subject. Such compositions may be specifically formulated for administration via one or more of a number of routes, including but not limited to, oral, parenteral, intravenous, intraarterial, subcutaneous, intranasal, sublingual, intraspinal, intracerebroventricular, and the like.

Suitable route of administration include, but are not limited to, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravesicularl, intravitreal, liposomal, local, mucosal, parenteral, rectal, subconjunctival, subcutaneous, sublingual, topical, transbuccal, transdermal, vaginal, in cremes, in lipid compositions, via a catheter, via a lavage, via continuous infusion, via infusion, via inhalation, via injection, via local delivery, or via localized perfusion. An “effective amount” or “therapeutically effective amount” of a composition, as used herein, is a predetermined amount calculated to achieve a desired effect.

In some examples, there is provided a method of using an ERO1 inhibitor (such as EN460 and/or T151742) to prevent pain. In a specific example, the method involves assessing the patient to determine if they are at risk of developing pain. If such risk is diagnosed, the patient is administered a therapeutically effective amount of ERO1 inhibitor (such as EN460 and/or T151742).

In some examples there is provided a combination therapy.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

An “oral dosage form” includes a unit dosage form prescribed or intended for oral administration. In an embodiment of the pharmaceutical combinations provided herein, the ERO1 inhibitor (e.g., EN460 and/or T151742) is administered as a unit dosage form.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

Abbreviations
endoplasmic reticulum (ER)
ER Oxidoreductin 1 (ERO1)
ER Oxidoreductin 1α (ERO1α)
ER-mitochondria contact sites (ERMCS)
dorsal root ganglia (DRGs)
mitochondrial calcium uniporter (MCU)
reactive oxygen species (ROS)
inositol 1,4,5 trisphosphate receptors (IP3Rs)
paw withdrawal threshold (PWT)
paraformaldehyde (PFA)
phosphate buffer (PB)
normal donkey serum (NDS)
Tetramethylrhodamine, Methyl Ester, Perchlorate (TMRM)
mitochondrial membrane potential (ΔΨm)
Propidium idiodide (PI)
N-methyl-D-glucamine artificial cerebrospinal fluid (NMDG-aCSF)
millivolts (mV)
hertz (Hz)
spinal dorsal horn (SDH)
monoamine oxidases (MAOs)

### Overview

Background: In the peripheral nervous system, nociceptors become hyperexcitable in both acute and chronic pain conditions. This phenotype can be mediated by dysregulated calcium, which occurs if the endoplasmic reticulum (ER) and mitochondria fail to buffer it appropriately. The redox enzyme endoplasmic reticulum oxidoreductin 1 (ERO1) regulates calcium transfer at ER-mitochondria contact sites (ERMCS). In this study we hypothesized that inhibiting ERO1 and thereby dampening ERMCS calcium transfer might lower nociceptor hyperexcitability in sensory neurons and pain-like behaviours in mice.

Methods: C57BL/6 mice were used for histology, behaviour, and cell culture experiments. Behaviour included thermal tail flick, the formalin hind paw injection model of acute inflammatory pain, and hind paw incision post-surgical pain. Post-mortem human dorsal root ganglia (DRGs) were used for immunohistochemistry and in vitro calcium imaging.

Results: Here we demonstrate that the α isoform of ERO1 is expressed in mouse dorsal root ganglia (DRGs) across multiple subtypes of mouse sensory neurons. This led us to peripherally administer an ERO1 inhibitor in mice, which acutely reversed nociception in acute inflammatory and post-surgical pain models. We hypothesized that this may be due to reduced excitability of DRG neurons, and tested ERO1 inhibition in vitro. In cultured DRGs, ERO1 inhibition dampened nociceptor excitability and mitochondrial function, suggesting that reduced calcium transfer through ERMCS could be responsible for the behaviour we observed in vivo. We also found ERO1α expression in human DRGs using immunohistochemistry and previously published single cell RNA sequencing data. Finally, we showed that ERO1 inhibition modulates human sensory neuronal excitability in cultured post-mortem DRGs.

Conclusions: We found that ERO1 inhibition dampens mitochondrial function, sensory neuron excitability, and acute pain-like behaviour in mice. Additionally, ERO1 inhibition decreases sensory neuron excitability in post-mortem human sensory neurons in vitro.

### 2. Materials and Methods

### 2.1 Sex as a biological variable

Sex was considered as a biological variable throughout this study. All mouse experiments were performed with consideration for both sexes. ERO1α histological colocalization, post-incisional behaviour, and cell culture experiments were performed with full sex-matching in parallel. Formalin experiments were continued in male animals after our initial experiment with both sexes failed to reveal statistically significant differences between the responses of males and females. The conditioned place preference experiment was performed in 3 male and 3 female animals per condition after seeing that EN460 transiently relieved mechanical post-incisional pain in both sexes. Human experiments included samples from both sexes, and data was pooled.

### 2.2 Mouse behaviour

All animal experiments were performed according to the Canadian Council on Animal Care’s Guidelines and Policies with ethics approval from the University of Alberta Health Sciences Animal Care and Use Committee (protocols 0000274 and 0002493). In total, 36 Male and 26 female naïve C57BL/6 mice were obtained for behavioural testing at 6-8 weeks of age from Charles River Canada and housed for at least one week prior to behavioural habituation. Animals were handled and habituated to behaviour apparatuses for two days prior to testing. For mouse intraperitoneal injections, EN460 (Millipore 328501) was dissolved in sterile 100% DMSO to a stock concentration of 25 mM and diluted in PBS to a final concentration of either 1, 3.13, 6.25, 10, 12.5, 15, or 25 mg/kg in 200 μL. Mice were treated with EN460 20-30 minutes prior to behavioural testing. Vehicle-treated groups received either 8.6%, 13.0%, or 29% DMSO corresponding to the EN460 concentration diluted in PBS. The potential for confounds was reduced by cycling the order of treatments and behavioural testing.

For the tail flick thermal behaviour test, mice were gently restrained in a soft conical tube. The distal 4 cm of the animal’s tail was immersed in 49 °C water, and the time to tail withdrawal was measured with a cut off time of 15 seconds to avoid tissue damage. All animals were habituated for two days prior to testing. Tail withdrawal baseline was measured 10 minutes prior to EN460 or vehicle injection, and 20 minutes following injection. The experimenter who performed the tail flick test was blinded to drug treatment.

Formalin testing was performed as previously described,²⁶ 6 animals were assigned to each group by an experimenter randomly at their discretion. EN460 or vehicle was injected intraperitoneally at either 10 or 25 mg/kg. After 20-30 minutes, 10 μL/g of 1% formalin was administered to the mouse hind paw. A second experimenter who was blinded to all treatments then scored nociceptive behaviour time. Time spent performing nociceptive behaviours was then scored for 30 minutes, in 5-minute intervals. Rotarod was performed by the blinded experimenter on this same cohort of animals both prior to EN460 and formalin injections, and 35 minutes after formalin injection (55 minutes after EN460 injection). Tissue was collected one hour after formalin injection to ensure peak c-FOS protein expression could be observed.^{26,27} When naloxone was added into the experiment it was administered to a new cohort of 4 animals per group (assigned to each group by an experimenter randomly at their discretion). at 1 mg/kg intraperitoneally 20-30 minutes prior to EN460 injection. The experimenter who scored the formalin behaviour was again blinded to all treatments.

Post-incisional pain was induced under isoflurane anesthesia by making a single incision into the left hind paw skin and muscle of the animal. 5 animals were assigned to each group. Mice were kept in their home cages and cages were assigned to either drug or vehicle groups at random by the experimenter at their discretion before the experiment began (and before baseline behaviour was assessed). This incision was closed with a single suture, which was not removed during the testing period. Daily injection of either 15 mg/kg EN460 or vehicle was administered intraperitoneally. Behavioural testing was performed 20-30 minutes after injection. Von Frey testing was performed as described in Friedman et al. 2023.28 0.01 – 2 g Von Frey filaments were used, employing the up-down method,29 where testing was initiated at 0.4 g of force and applied for 5 seconds or until the mouse responded. Filaments of increasing force were then used if a response was absent, and filaments of decreasing force were used if a response was present. Three responses were used to calculate a 50% paw withdrawal threshold (PWT).³⁰ Hargreaves testing was performed with an Ugo Basile laser at an intensity of 72 and averaged across three measurements per paw, per animal, with a cut-off of 15 seconds to prevent tissue damage.³¹ The experimenter who performed behavioural testing collected data was blinded to vehicle vs. EN460 treatment groups as these injections were performed by a different experimenter.

A second cohort of post-incisional animals (6 males and 6 females) was assessed using a conditioned place preference paradigm. Animals were housed in cages of three and one cage of each sex was randomly assigned by an experimenter to either the anesthesia control or incisional groups. The remainder of the test was performed by another experimenter. Animals were habituated and handled in the room for two days prior to being allowed to explore the chambers openly. EN460 injections were paired to the least-preferred chamber if one was identified in the baseline. Animals then received alternating vehicle and EN460 injections twice daily, separated by at least four hours. Drug and vehicle treatment timing (morning or afternoon) was also rotated to reduce confounds. On day 6 animals were allowed to roam free, and on day 7 they were allowed to roam free 20 minutes after EN460 injection. Behaviour was recorded then analyzed with EthoVision video tracking software.

### 2.3 Mouse dorsal root ganglia immunohistochemistry (IHC)

Animals were euthanized with 0.1 mL sodium pentobarbital and perfused with 30 mL of 0.9% saline followed by 20 mL of 4% paraformaldehyde (PFA) in 0.1M phosphate buffer (PB) pH 7.4. Tissue was dissected then drop-fixed in PFA overnight, followed by two days in 30% sucrose in 0.1M PB. Tissue was frozen in OCT medium and cryo-sectioned to 10 um thickness. Slides were incubated with antigen retrieval solution (1.92 g anhydrous citric acid and 0.5 mL Tween20 in 1 L H2O, pH 6.0) for 10 min before washing in PBS followed by PBS with 0.2% triton X-100 in PBS (PBSTX). Tissue was blocked for one hour at room temperature in 10% normal donkey serum (NDS) in PBSTX. Primary antibody was incubated overnight at 4 °C in 2% NDS and 2% bovine serum albumin (BSA) in PBSTX. Primary antibodies included ERO1L (1:500, Abcam EPR12474), P2X3 (1:50, Neuromics GP10108), NF200 (1:5000, ThermoFisher PA1-10002), and c-FOS (1:500, Cell Signaling #2250). The next day, slides were washed twice in PBS_{TX} and once in PBS before incubation with secondary antibodies (Jackson Immunoresearch) at a 1:200 dilution for 45 min. Slides were washed again twice in PBS_{TX} and once in PBS before mounting with fluoromount G^{TM} Mounting Medium (Invitrogen 00-4958-02). Slides were imaged at 20x magnification for analysis using a Zeiss AxioImager. Images were analyzed using Fiji ImageJ. For ERO1α antibody co-staining, tissue from 10 male and 8 female animals was used. For c-FOS antibody staining, tissue was collected from the 12 male animals used in the formalin experiment, one hour after formalin injection as previously described.^{26,27}

### 2.4 Mouse dorsal root ganglia cultures

DRG dissociation and culture protocol was adapted from Maguire et al. 2022.³² Two 8-10 week old C57/BL6 mice of each sex were used per experiment. Animals were euthanized with 0.1 mL sodium pentobarbital and perfused with 30 mL cold 0.9% saline then all DRGs were collected in HBSS-/- (Gibco 14175-095) on ice. Tissue was digested in 2 mg/mL Stemxyme I (Worthington LS004106) and 0.01% DNAse (Worthington LS002007) for 1hr in a 37°C water bath. Cells were gently triturated and centrifuged for 10 minutes at 300 xg. Cells were resuspended in 0.02% bovine serum albumin (BSA, Sigma Aldrich A4161) and 0.01% DNAse in DPBS then passed through a 70 μm cell strainer. Cells were then layered on a 15% BSA cushion (Sigma Aldrich A7906) and centrifuged for 10 minutes at 300 xg with slow acceleration and deceleration to remove myelin debris. Finally, cells were counted and plated at 500 cells per well in 96 well imaging plates (Falcon 353219). Media was composed of 1:1 DMEM (Gibco 11960069): Neurobasal (Gibco 21103049), supplemented with SATO (Sigma Aldrich Progesterone P8783, Sodium Selenite S5261, Transferrin T1147, Putrescine dihydrochloride P5780), N-acetylcysteine (Sigma Aldrich A8199), insulin (Sigma Aldrich I2643), penicillin/streptomycin (Gibco 15140-122), B27+ (Gibco A3582801), GlutaMax (ThermoFisher 35050061), and sodium pyruvate (ThermoFisher 11360070). Cells were maintained in culture for 48 hours.

For in vitro treatment, EN460 was diluted from 25 mM stock to a final concentration of 10 μM in cell culture media. In imaging experiments EN460 was either added upon plating and remained for the culture period or added directly before imaging. For electrophysiology, EN460 was added to media approximately one hour after plating and remained for the entirety of the culture period. Cells were removed at 6, 12, 24, or 48 hours on coverslip fractions for analysis. Another ERO1 inhibitor, T151742, was diluted in DMSO to a 25mM stock as well, and used at a final concentration of 5 μM. It was provided by the Hazlehurst lab at West Virginia University.³³

### 2.5 Electrophysiology

For electrophysiological analysis, DRG neurons were plated at 1000 cells per well in 24 well plates on 15mm glass coverslips. 10 μM EN460 or 5 μM T151742 was added shortly after plating for either 6, 12, 24, or 48 hours before analysis. For measurement of action potentials from mouse DRG neurons, recordings were generated in current-clamp mode using an Axopatch 200B amplifier (Molecular Devices) to assess DRG neuron excitability in both untreated (control) conditions, and drug-treated conditions at the four timepoints listed. Electrodes had a tip resistance of 3-6 MΩ when filled with pipette solution. The external (bath) recording solution had the following composition (in mM): 135 NaCl, 5 KCl, 1 CaCl2, 1 MgCl2, 10 HEPES and adjusted to pH 7.4 with NaOH. The internal (pipette) recording solution had the following composition (in mM): 135 KCl, 4 Na2-ATP, 5 K-EGTA, 10 HEPES and adjusted to pH 7.2 with KOH. Protocols used to elicit action potentials included 10 pA or 100 pA (3s) stepwise current injections, current ramps, or continuous (gap-free) observations of spontaneous action potentials with no (0 pA) current injections, to measure features of neuronal excitability. These parameters include, the resting membrane potential of the neuron, action potential (spike) firing frequency, and the rheobase (minimum threshold of current injection required to generate an action potential). All data was acquired using clampex software hen manually analyzed using Clampfit10.7 (Molecular Devices). The resting membrane potential was assessed at pA=0, immediately following whole cell break in of the neuron. The frequency of action potentials was analyzed using the event detection feature of Clampfit 10.7.

### 2.6 In vitro staining

Live staining for mitochondrial membrane potential (ΔΨm) was performed with Tetramethylrhodamine, Methyl Ester, Perchlorate (TMRM). TMRM is a fluorescent indicator that accumulates more within the mitochondria matrix when the potential gradient across the membrane is higher (i.e. the interior is more negatively charged).34 100 nM TMRM was incubated for 30 minutes at 37 °C, with 1:200 NucBlue (1:200 Invitrogen R37605) for the final 10 minutes. Cells were then washed and placed in fresh media. In addition to cells treated for 48 hours with EN460, untreated wells were imaged at baseline, then treated with 10 μM EN460, and imaged every 5 minutes to capture the acute effect of the drug.

Mitochondrial ROS was assessed with MitoTracker Orange CM-H2TMRos (1: 10 000, Molecular Probes M7511) for 30 minutes at 37 °C, with 1:200 NucBlue for the final 10 minutes. Cells were fixed with a 4% final concentration of paraformaldehyde in 0.1 M PB for 15 minutes at room temperature, then washed three times with D-PBS. Cells were imaged in D-PBS.

Cell death was stained for with Propidium Iodide (PI) (1:20 Invitrogen P3566) and 1:200 NucBlue for 15 minutes at 37 °C. Cells were then washed once with warm D-PBS, and wells were flooded with warm media for live imaging.

All culture images were acquired on an ImageXpress Micro high content screening system, courtesy of Dr. Sun and the Cross Cancer Institute at the University of Alberta.

### 2.7 Seahorse

Mitochondrial and glycolytic ATP production were assessed using the XF Real-Time ATP Rate Assay (Agilent 103591-100). The assay was performed according to instructions using a Seahorse XF HS mini. DRG cultures were plated in XF HS miniplates (Agilent 103725-100) at 1500 cells per well either with or without 10 μM EN460 for 48 hours. An assay cartridge was hydrated overnight prior to assay. Cells were degassed prior to assay for one hour in a non-CO2 incubator at 37 °C in assay media (Agilent 103680-100).

### 2.8 Human dorsal root ganglia calcium imaging

Human DRGs (Table 1) were obtained from organ donors and cultured according to a previously published protocol.^{35,36} In brief, human DRGs were immersed in N-methyl-D-glucamine artificial cerebrospinal fluid (NMDG-aCSF).37 DRGs were cut into 1 mm chunks and dissociated in pre-warmed (37°C) 2 mg/ml STEMxyme I (Worthington LS004106) and 4 µg/ml DNAse (Worthington LS002139) for 4 hours in a shaking water bath. Tissue was further triturated every hour using glass pipettes. Dissociated cells were plated on 12 mm glass coverslips coated with 100 µg/ml poly-D-lysine (Sigma P7405). Cells were maintained in BrainPhys media (STEMCell 05790) supplemented with 2% SM1 (STEMCell 05711), 1% N2-A (STEMCell 07152), 1% PenStrep (ThermoFisher 15070063), and 1% GlutaMax (Gibco 35050061) at 37°C and 5%CO₂ for at least 24 hours and up to 48 hours prior to calcium imaging.

EN460 was prepared as a 25 mM stock in 100% DMSO and was diluted to 10 µM in human DRG BrainPhys media. Human DRG neurons were treated with 10 µM EN460 for 30 minutes by completely replacing the media with media plus EN460 or vehicle. Vehicle was 0.04% DMSO in human DRG BrainPhys media. After 30 minutes of drug treatment, the media was replaced by a calcium sensitive dye.

Calcium transients were recorded using Fluo4-AM (ThermoFisher F14201). A 500 µM Fluo4-AM (50 µg) stock was made by diluting it in HBSS without Ca²⁺ and Mg²⁺ (ThermoFisher 14170161) plus 2% Pluronic F-127 (ThermoFisher P3000MP). Fluo4-AM stock was further dissolved (1:200) in HBSS without Ca²⁺ and Mg²⁺ for a final concentration of 2.5 µM. Human DRG neurons were incubated in 1 ml of Fluo4-AM-HBSS solution for 30 min at 37°C and 5%CO₂. Calcium imaging was performed on Olympus IX83 Inverted Microscope equipped with Elveflow OB1 microfluidics system (Elvesys) fitted with RC25 coverslip recording chamber (Harvard Apparatus). Imaging was performed at 32 ̊C using a 20X air objective on MetaFluor imaging software (Molecular Devices). Human DRG neurons were perfused with a pre-warmed and bubbled bath-aCSF (in mM: 121 NaCl, 4.2 KCl, 1.1 CaCl₂, 1 MgSO₄, 29 NaHCO₃, 0.45 NaH₂PO₄-H₂O, 0.5 Na₂HPO₄, 20 glucose) with a pH of 7.4 and osmolarity of 300 mOsm. Fluorescence of each cell was normalized to the average fluorescence of the first 30 seconds. Amplitude of high KCl (50mM) response was calculated by subtracting the average fluorescence of the 30 seconds prior to KCl treatment and the maximum fluorescence after the stimulation (Fₘₐₓ - F_{BL}).

### 2.9 Human dorsal root ganglia immunohistochemistry

Human DRGs were cryo-sectioned at 20 microns onto charged slides and then fixed in 10% formalin (ThermoFisher 23-245684) for 5 minutes, then washed in PBS twice. Each section was at least 100 microns apart to prevent sampling from the same neuron. For antigen retrieval, slides were immersed in citric acid buffer (1.92 g anhydrous citric acid in 1L water and 0.5 ml Tween-20, pH adjusted to 6.0 with NaOH) and heated to 55 °C for 15 minutes. Slides were then washed in PBS (2 x 10 minutes) and PBS with 0.1% Tween-20 (1 x 10 minutes). Tissue was blocked using a solution of 10% normal goat serum (R&D Systems S13150H) in PBS with 0.1% TritonX-100 for 1 hour at room temperature. Primary antibodies were diluted in antibody solution (200µL normal goat serum, 9800µL PBS with 0.1% TritonX-100, and 200mg bovine serum albumin (BioPharm 71-015-025)) and incubated at 4°C overnight. Slides were washed in PBS (2 x 10 minutes) and PBS with 0.1% Tween-20 (1 x 10 minutes). Secondary antibodies were diluted in antibody solution and incubated for 1 hour at room temperature. DAPI was diluted in PBS and incubated for 5 minutes. TrueBlack Lipofuscin quencher (Biotium 23007) was diluted in 70% ethanol and incubated for 30 seconds per slide. Slides were washed in PBS (2 x 10 minutes) and PBS with 0.1% Tween-20 (1 x 10 minutes). Slides were mounted with ProLong Gold Antifade Mountant (Fisher Scientific, P36930) and coverslips. Primary antibodies used were chicken anti-peripherin (EnCor CPCA-Peri, 1:1000), and rabbit anti-ERO1L antibody (Abcam ab177156, 1:200). Secondary antibodies used are goat anti-chicken 488 (ThermoFisher A-11039, 1:2000), and Thermofisher goat anti-rabbit 555 (ThermoFisher A21428, 1:2000). Human DRGs were imaged on Olympus FV1200 at 20X magnification using the FluoView imaging software. Images were analyzed using CellSens (Olympus). Fluorescence levels were quantified by circling neurons in a single field of view.

### 3. Statistical analyses

### 3.1 Mouse behaviour

4-6 animals were used per group based on power calculations with an estimated effect size 0.65, type I error level and power set at 5% and 80% respectively. No animals were excluded from calculations, though outlier analysis was performed using the ROUT method. All graphs and statistics were generated using GraphPad Prism 10.4.0 software. We confirmed normality using Kolmogorov-Smirnov tests for each ANOVA (α=0.05).

The primary outcome measure for the tail flick test was time to tail withdrawal, this is expressed in seconds. The experimental unit was a single animal, and the groups compared were vehicle vs. drug treated (n=7 animals per group). The median effective dose (ED50) was calculated to be 15 mg/kg.

The primary outcome measure for the formalin test was nociceptive behaviour time, this is expressed in each graph in seconds per 15-minute block of total time, divided into phase I (0-15 minutes) and phase II (15-30 minutes). The experimental unit was a single animal, and the groups compared were vehicle vs. drug treated (n=6 animals per group in the formalin and rotarod 10 mg/kg EN460 cohort, n=5-7 animals per group in the formalin 25mg/kg EN460 cohort, and n=4 animals per group in the formalin + naloxone 10 mg/kg EN460 cohort). Data was analyzed by Repeated Measures Two-way ANOVA with Sidak’s multiple comparisons, and significance was set at p<0.05. For rotarod data, the primary outcome measure time spent on the apparatus, which was expressed in minutes and the groups compared were vehicle vs. drug treated. Data points represent an average across 6 male animals. Data was analyzed by Repeated Measures Two-way ANOVA with Sidak’s multiple comparisons, and significance was set at p<0.05. Sphericity was assumed given that the experimental design relies on the matching of two conditions rather than repeated measurements across three or more conditions.

The primary outcome measure for Von Frey data average filament weight (in grams) required to induce a nociceptive response, this was expressed as 50% paw withdrawal threshold (PWT). The experimental unit was a single animal, and the groups compared were vehicle vs. drug treated. In the Hargreaves test, the primary outcome measure was time required to induce paw withdrawal, which was expressed on the graph in seconds. The experimental unit was a single animal, and the groups compared were vehicle vs. drug treated. Each data point represents one animal. Data was analyzed by Repeated Measures Two-way ANOVA with Sidak’s multiple comparisons, and significance was set at p<0.05. n=5 animals per group. Sphericity was assumed given that the experimental design relies on the matching of two conditions rather than repeated measurements across three or more conditions.

### 3.2 c-FOS immunohistochemistry

The primary outcome measure for this stain was the number of stained cells in the dorsal horn of the spinal cord. The experimental unit was a single animal, and the groups compared were vehicle vs. drug treated. No animals were excluded from analysis. Positive cells were manually counted and normalized to total DRG area in mm². Each data point represents one animal, n=6 animals per group. Data was analyzed by unpaired t-test, and significance was set at p<0.05. This data passed the Shapiro-Wilk normality test (vehicle p=0.20, EN460 p=0.10). We confirmed normality using the Kolmogorov-Smirnov test (α=0.05). All graphs and statistics were generated using GraphPad Prism 10.4.0 software.

### 3.3 Electrophysiology

The primary outcome measures were the rheobase, resting membrane potential, and action potential frequency as indicators of cell excitability. The experimental unit was a single cell, and the groups compared were vehicle vs. drug treated. Resting membrane potential was expressed in millivolts (mV), and rheobase was expressed in picoamperes (pA). Each data point represents one cell, collected across 5-10 experiments. Data was analyzed by Two-way ANOVA with Sidak’s multiple comparisons, and significance was set at p<0.05. Action potential Frequency was expressed in hertz (Hz). Data points represent an average across 17-31 cells per group, collected across 5-10 experiments. Data was analyzed by student’s t test between control and drug-treated groups for each current injection. No formal sample size calculations were performed. All in vitro mouse DRG electrophysiology experiments were performed with the maximum number of cells that could be assessed from at least 5 experiments. This resulted in n=17-120 cells being included for analysis depending on the measurement. Cells were only excluded from calculations if identified using outlier analysis, which was performed using the ROUT method. We confirmed normality using Kolmogorov-Smirnov tests for each ANOVA (α=0.05). All graphs and statistics were generated using GraphPad Prism 10.4.0 software.

### 3.4 In vitro mouse dorsal root ganglia staining

For TMRM staining, the primary outcome measure was signal intensity per cell, as an indicator of ΔΨm. The experimental unit was a single well, and the groups compared were vehicle vs. drug treated. Acute data was expressed as fold change of stain fluorescent intensity, normalized to respective baselines, and plotted over time. Data points represent an average across 8 wells from two experiments. Sphericity was assumed given that the experimental design relies on the matching of two conditions rather than repeated measurements across three or more conditions. This data was also expressed as fold change of area under the curve of integrated intensity from 0-40 minutes normalized to sex-respective untreated wells, where each data point represents one well (n=8 from two experiments with 4 wells each). Chronic data was expressed as fold change of integrated intensity normalized to sex-respective untreated wells, where each data point represents one well (n=16 from four experiments with 4 wells each). All data was analyzed by Two-way ANOVA (Repeated Measures for the acute timeline) with Sidak’s multiple comparisons, and significance was set at p<0.05.

For MitoTracker staining, the primary outcome measure was the percentage of cells positive for the stain, as an indicator of cells producing ROS. The experimental unit was a single well, and the groups compared were vehicle vs. drug treated. Data was expressed as fold change of percentage of NucBlue-positive cells co-stained with MitoTracker normalized to sex-respective untreated wells. Each data point represents one well (n=8 from two experiments with 4 wells each). Data was analyzed by Two-way ANOVA Sidak’s multiple comparisons, and significance was set at p<0.05.

For propidium iodide staining, the primary outcome measure was the percentage of cells positive for the stain, as an indicator of dead cells. The experimental unit was a single well, and the groups compared were vehicle vs. drug treated. Data was expressed as fold change of percentage of NucBlue-positive cells co-stained with PI normalized to sex-respective untreated wells. Each data point represents one well (n=12 from three experiments with 4 wells each). Data was analyzed by Two-way ANOVA with Sidak’s multiple comparisons, and significance was set at p<0.05.

No formal sample size calculations were performed. All in vitro mouse DRG staining experiments were performed with at least four wells per group and pooled across at least two experiments. Data points were only excluded from calculations if identified using outlier analysis, which was performed using the ROUT method. We confirmed normality using Kolmogorov-Smirnov tests for each ANOVA (α=0.05). All graphs and statistics were generated using GraphPad Prism 10.4.0 software.

### 3.5 Seahorse

The primary outcome measure for the seahorse experiments was oxygen consumption and extracellular acidification over time as indicators of mitochondrial and glycolytic ATP production respectively. The experimental unit was a single well, and the groups compared were vehicle vs. drug treated. Data was expressed as fold change of picomoles / minute/ total cells normalized to sex-respective untreated wells. Each data point represents one well (n=9 from three experiments with 3 wells each). Data was analyzed by Two-way ANOVA with Sidak’s multiple comparisons, and significance was set at p<0.05. No formal sample size calculations were performed. All in vitro mouse DRG seahorse experiments were performed with at three wells per group and pooled across at least three experiments. Data points were only excluded from calculations if identified using outlier analysis, which was performed using the ROUT method. We confirmed normality using Kolmogorov-Smirnov tests for each ANOVA (α=0.05). All graphs and statistics were generated using GraphPad Prism 10.4.0 software.

### 3.6 Human dorsal root ganglia immunohistochemistry and calcium imaging

For human DRG immunohistochemistry, DRG neurons were identified by staining with an antibody for peripherin. The primary outcome measures were the cell body size a ERO1α antibody staining intensity. The experimental unit was a single cell, and no groups were compared. These measurements were plotted against each other to determine whether a particular size of neuron appeared to express more ERO1α protein. No cells were removed from analysis, n=228 cells.

The primary outcome measure for human DRG calcium imaging was the fluorescent indicator response to KCl depolarization as an indicator of cell excitability. The experimental unit was a single cell, and the groups compared were vehicle vs. drug treated. Data was expressed as amplitude of fluorescent response to KCl. Each data point represents one neuron, collected across three experiments. Data was analyzed by unpaired t-test, and significance was set at p<0.05. No formal sample size calculations were performed. As many cells as possible were recorded per experiment, across three experiments, n=55 vehicle-treated cells, and 43 EN460-treated cells. Cells that did not respond to KCl or had less than 10% response to KCl as compared to baseline were not included in analysis. We confirmed normality using the Kolmogorov-Smirnov test (α=0.05).

### 4. Results

### 4.1 Mouse dorsal root ganglia express ERO1α across three main neuronal subtypes.

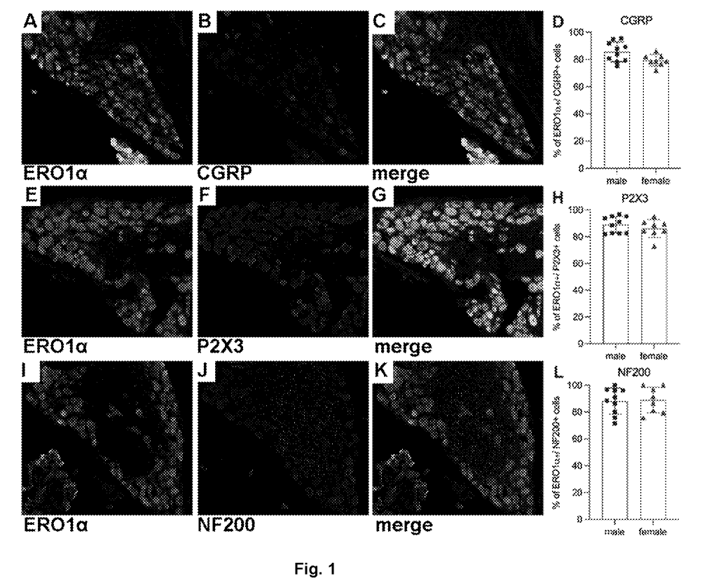

To understand if ERO1 could be a viable target to treat pain we first sought to determine if it was expressed in peripheral sensory neurons of the DRG. We performed immunohistochemistry on DRGs to probe for the ERO1α isoform, which is known to be expressed ubiquitously, whereas the other isoform ERO1β is concentrated to stomach and pancreatic tissues.^{38,39} In CGRP+ cells, representative of peptidergic nociceptors,40 86% ±7 of male neurons, and 79% ±4 of female neurons robustly expressed ERO1α (FIG. 1A-D). In P2X3+ cells, representative of the non-peptidergic class of nociceptors,⁴⁰ 89% ±6 of male neurons, and 86% ±7 of female neurons expressed ERO1α (FIG. 1E-H). Finally, in NF200+ neurons, representative of both small and larger myelinated A fibre cell bodies,^{41,42} 88% ±10 of male neurons, 89% ±10 of female neurons expressed ERO1α (FIG. 1I-L). These findings indicate that ERO1α is expressed in most sensory neurons of the DRG.

### 4.2 ERO1 inhibition transiently lowers acute inflammatory pain responses in mice.

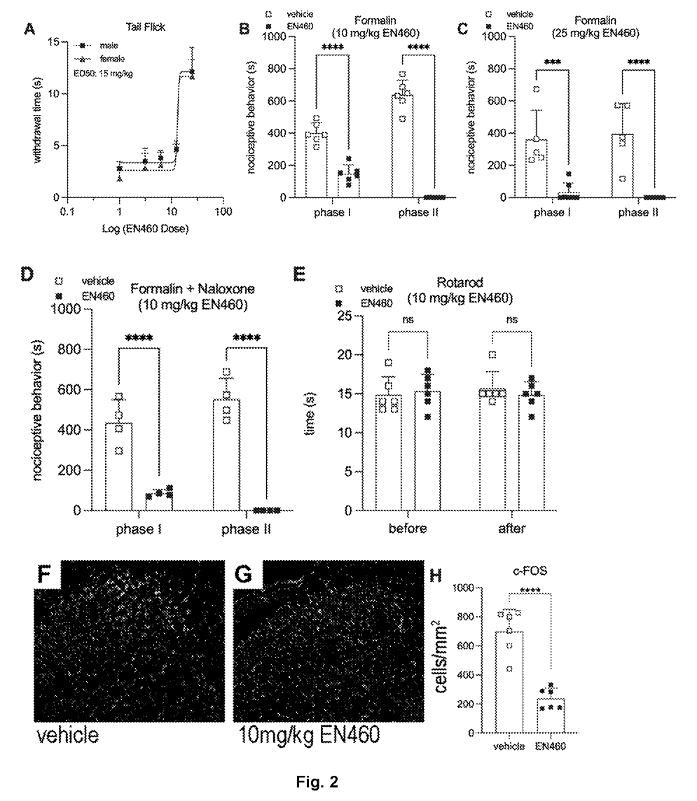

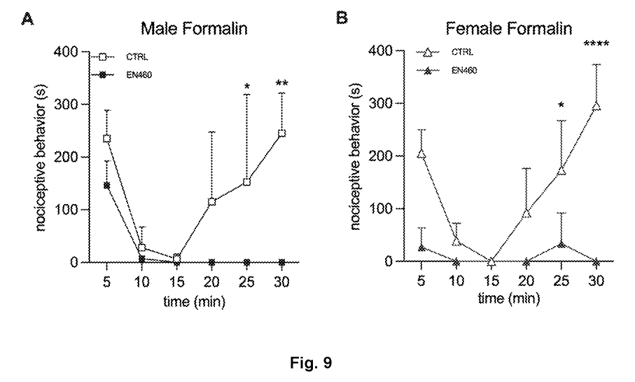

We next sought to investigate whether ERO1 inhibition could provide an analgesic effect. This was first tested by administering the ERO1 inhibitor EN460 to naïve mice in a thermal tail flick test, which revealed that ERO1 inhibition with EN460 increased withdrawal time with an ED50 of 15 mg/kg (FIG. 2A). Next, we utilized an acute inflammatory pain test, wherein we injected formalin into the hind paw of mice pre-treated with 10 mg/kg EN460, then recorded the time they spent performing nociceptive/ nocifensive behaviours such as paw guarding, shaking, and licking. In our initial experiment involving both sexes we did not find any statistically significant sex differences (FIG. 9) (5 minutes p=0.12, 10 minutes p>0.99, 15 minutes p>0.99, 20 minutes p>0.99, 25 minutes p=0.92, 30 minutes p>0.99, Tukey’s multiple comparisons test, Repeated Measures Two-way ANOVA). We followed this experiment with another three cohorts of mice to determine if the effect of EN460 could be due to motor impairment or off-target opioid involvement.

In the second cohort we observed nociceptive behaviours in response to formalin injection were dramatically attenuated in mice treated with 10 mg/kg EN460 (FIG. 2B) (vehicle mean=519 ±145, EN460 mean=74 ±85, F(1,10=331, p<0.0001, d=1, 95% CI 391 to 499, Repeated Measures Two-way ANOVA). In the first phase of the formalin response (0-15 min) there was a significant reduction in nociceptive behaviour with EN460 treatment. Strikingly, this was followed by a complete abolition of second phase behaviours at 15-30 minutes in mice treated with EN460 compared to vehicle. (phase I: vehicle mean=400 ±64, EN460 mean=148 ±55, p<0.0001. phase II: vehicle mean =638 ±92, EN460 mean=0 ± 1, p<0.0001, Uncorrected Fisher’s LSD). In the third cohort of mice, we tested the highest dose used in the tail flick, 25 mg/kg EN460 to determine if we could abolish first phase behaviours. Again, we found that nociceptive behaviour was attenuated over all (vehicle mean=377 ±176, EN460 mean=16 ±43, F(1,10=51.45, p<0.0001, d=1, 95% CI 249 to 472.7, Repeated Measures Two-way ANOVA). First phase behaviours were almost completely attenuated with this higher dose of EN460. and second phase behaviours remained abolished as well (phase I: vehicle mean=360 ±182, EN460 mean=33 ±58, p<0.0001. phase II: vehicle mean =394 ±190, EN460 mean=0 ±0, p<0.0001, Uncorrected Fisher’s LSD).

To determine if the profound analgesia we observed with EN460 was being mediated by an endogenous opioid mechanism, we repeated the formalin test in a third cohort of mice with a pre-treatment of the opioid receptor antagonist naloxone at 1 mg/kg (FIG. 2B). Pre-treatment with naloxone however, had no impact on the analgesic potency of EN460 (vehicle mean=494 ±119, EN460 mean=43 ±48, F(1,6)=184.8, p<0.0001, d=1.0, 95% CI 370 to 532, Repeated measures Two-way ANOVA. Phase I: vehicle mean=436 ±114, EN460 mean= 86±19, p=0.0048. Phase II: vehicle mean=552 ±190, EN460 mean=0 ±0, p=0.0004. Uncorrected Fisher’s LSD). These results suggest that the mechanism by which EN460 reduces pain-like behaviours in mice is independent from opioid receptor activation.

To determine if the abolition of formalin-induced nociceptive behaviour was due to off target motor impairments in response to EN460, we performed a rotarod test on the second cohort of formalin-treated animals before treatment and after drug and formalin treatment once nociceptive behaviours ceased (FIG. 2C). We saw no significant differences in performance after treatment with EN460 (before mean=15 ±2, after mean=15 ±2, F(1,10)=0.01980, p=0.8909, d=0.0019, 95% CI -2 to 2, Repeated Measures Two-way ANOVA), or when directly comparing vehicle to EN460 treatment at the after timepoint (vehicle mean=16 ±2, EN460 mean=15 ±2, p=0.75, Sidak’s multiple comparisons test).

We hypothesized that EN460 dampens primary nociceptor excitability and nociceptive transmission, and to support this we collected spinal cord tissue from the formalin-treated animals, as primary nociceptors synapse in the spinal dorsal horn (SDH). We then used immunohistochemistry to examine the expression levels of the neuronal activation marker c-FOS as an indicator of post-synaptic activity in response to formalin stimulation (FIG. 2D, E). EN460 treatment significantly reduced the number of c-FOS positive SDH cells compared to vehicle treated mice (FIG. 2F) (vehicle mean=698.60 ±152.60, EN460 mean=237.90 ±72.08, F(5,5)=4.48, p<0.0001, d=3.86, 95% CI -614.3 to -3, Unpaired t test). Collectively, these results suggest that ERO1 inhibition with EN460 decreases nociceptive behaviours through a non-motor, non-opioid mechanism, likely due to reduced activation of sensory neurons.

### 4.3 ERO1 inhibition acutely reverses mechanical hypersensitivity in a mouse model of post-surgical pain.

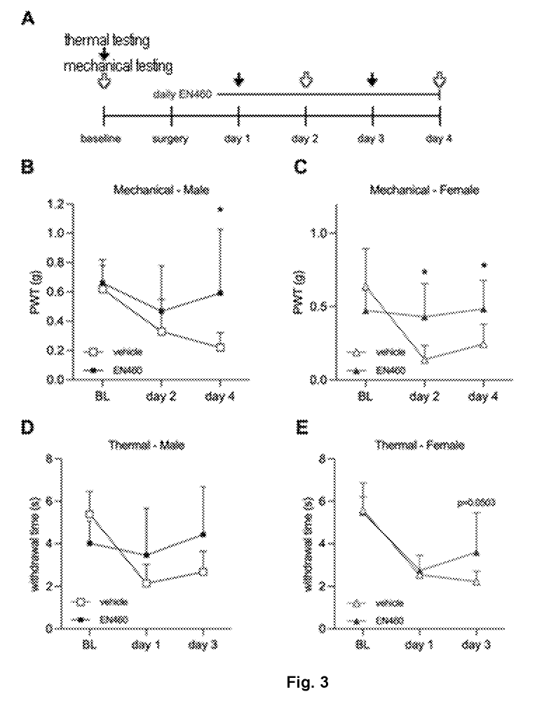

Having found that ERO1 inhibition reduced nociceptive behaviours in the formalin model, we next sought to determine if inhibiting ERO1 would affect nociception in a more clinically relevant pain model. We used an incisional model of post-surgical pain,43 and treated mice with EN460 daily for four days after surgery. Mechanical and thermal nociceptive thresholds were assessed 30 minutes after injection of EN460 (FIG. 3A).

On day 4 post-surgery male mice displayed a nociceptive response to lower weight Von Frey filaments, indicative of mechanical hypersensitivity, (vehicle baseline vs. day 4, baseline mean=0.62 ±0.20, day 4 mean=0.22 ±0.10, p=0.035), however EN460 acutely reversed this hypersensitivity (vehicle vs. EN460, vehicle mean=0.22 ±0.10, EN460 mean=0.59 ±0.43, p=0.031, Sidak’s multiple comparisons test, Repeated Measures Two-way ANOVA). Female animals displayed mechanical hypersensitivity on day 2 (baseline vs. day 2, baseline mean=0.64 ±0.26, day 2 mean=0.14 ±0.095, p=0.0011) and day 4 (baseline vs. day 4, baseline mean=0.64 ±0.26, day 2 mean=0.24 ±0.14, p=0.0073), which was reversed by EN460 (day 2 vehicle mean=0.14 ±0.10, EN460 mean=0.43 ±0.23, p=0.020. day 4 vehicle mean=0.24 ±0.14, EN460 mean=0.48 ±0.19, p=0.049, Sidak’s multiple comparisons test, Repeated Measures Two-way ANOVA) (FIG. 3B, C).

Responses to noxious heat in the Hargreaves test did not reveal an effect of EN460 treatment in either sex. Animals developed heat hypersensitivity on days 1 and 3 (Male vehicle baseline vs. day 1, baseline mean=5.38 ±1.08, day 1 mean=2.14 ±0.89, p=0.0010. Male vehicle baseline vs. day 3, baseline mean=5.38 ±1.08, day 3 mean=2.68 ±0.97, p=0.0046. Female vehicle baseline vs. day 1, baseline mean=5.58 ±1.29, day 1 mean=2.55 ±0.92, p<0.0001. Female vehicle baseline vs. day 3, baseline mean=5.58 ±1.29, day 3 mean=2.23 ±0.49, p<0.0001, Sidak’s multiple comparisons test, Repeated Measures Two-way ANOVA). However, inhibition of ERO1 with EN460 had no statistically significant effect on these measurements (Male EN460 baseline vs. day 1, baseline mean=4.02 ±1.05, day 1 mean=3.47 ±2.19, p=0.0010. Male EN460 baseline vs. day 3, baseline mean=4.02 ±1.05, day 3 mean=4.43 ±2.25, p=0.0046. Female EN460 baseline vs. day 1, baseline mean=5.48 ±0.73, day 1 mean=2.47 ±0.16, p<0.0001. Female EN460 baseline vs. day 3, baseline mean=5.48 ±0.73, day 1 mean=3.61 ±1.85, p<0.0001, Sidak’s multiple comparisons test, Repeated Measures Two-way ANOVA) (FIG. 3D, E).

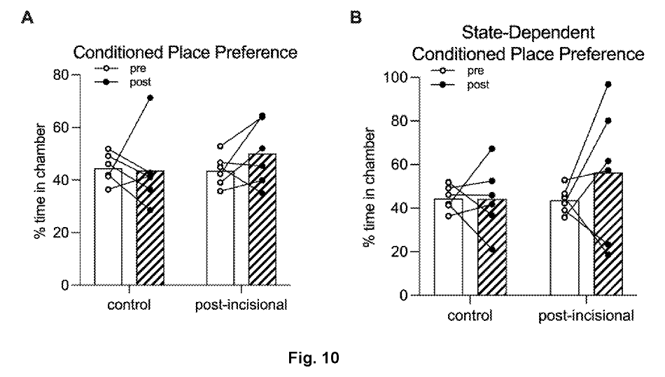

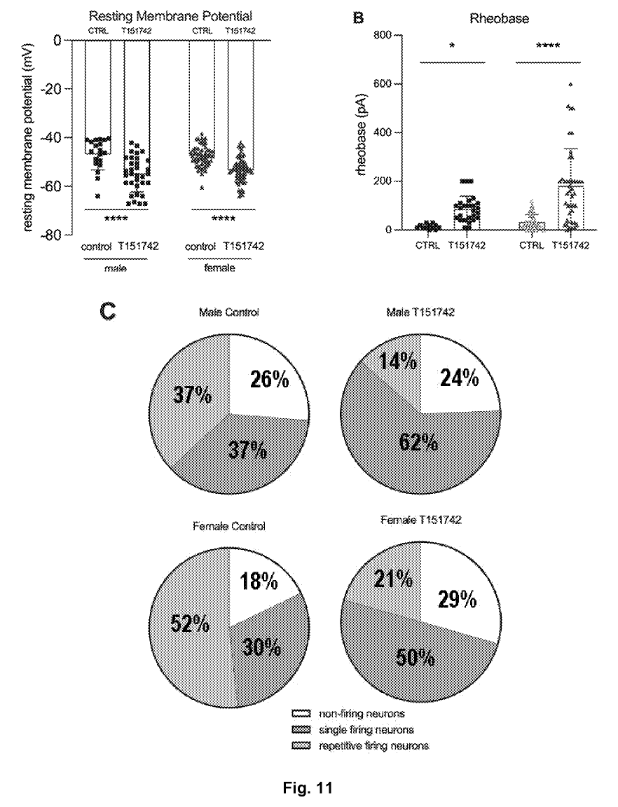

Lastly, in a separate cohort of mice, we performed a conditioned place preference assay in control (anesthesia, but no incision) and post-incisional animals using 15 mg/kg EN460 to determine whether EN460 in intrinsically rewarding in the control animals (FIG. 10). No preference was observed for either saline or drug-paired chambers in naïve or post-incisional animals (FIG. 10A) (control mean=44.06 ±10.52, post-incisional mean=46.85 ±9.84, F(1,10)=0.49, p=0.50, d=0.047, 95% CI -11.90 to 6.21, Repeated Measures Two-way ANOVA). The next day, animals were given EN460 to test for a state-dependent preference and again no statistically significant effect was observed (FIG. 10B) (control mean=44.36 ±11.10, post-incisional mean=49.96 ±22.16, F(1,10)=0.79, p=0.40, d=0.73, 95% CI -21.94 to 9.45, Repeated Measures Two-way ANOVA). This lack of preference in the control animals suggests that EN460 is not intrinsically rewarding and is not likely to be addictive. The lack of EN460 preference in post-incisional animals may indicate that the rewarding effect of the drug during acute pain-like conditions is too minor to be captured by this assay, or that the effect may stronger during an earlier phase when the incisional nociceptive effects are more acute.⁴⁴

### 4.4 Mouse sensory neurons are less excitable in vitro after ERO1 inhibition.

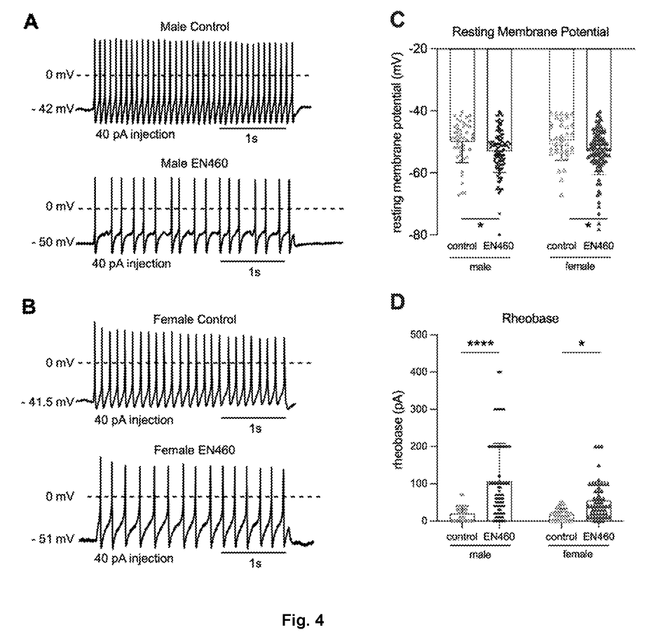

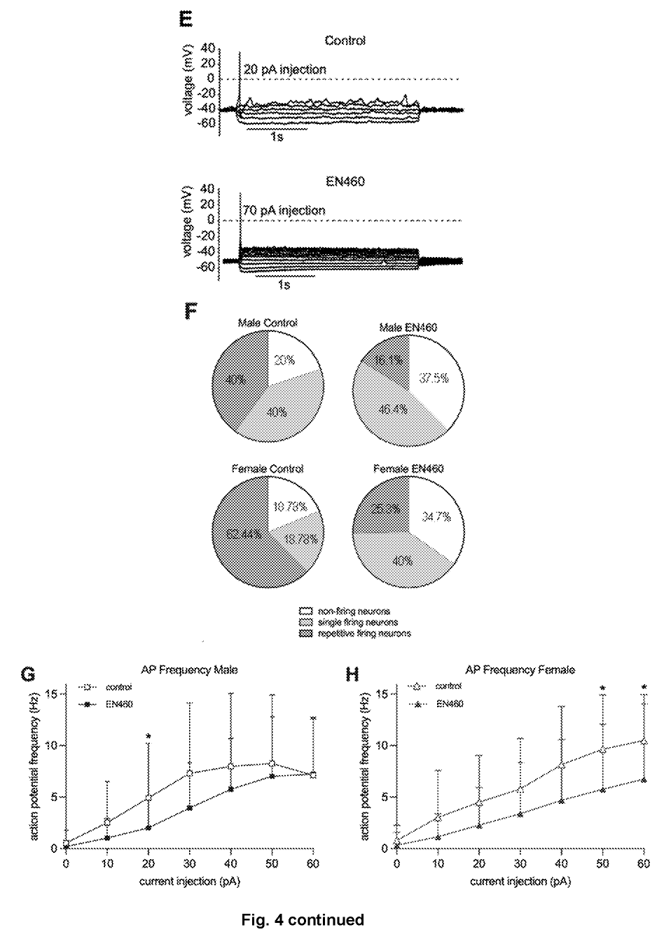

Next, we tested whether ERO1 inhibition could lower DRG neuron excitability in vitro using whole cell patch clamp electrophysiology. First, we observed that the resting membrane potential (FIG. 4A-C, E) of EN460-treated cells was lower than untreated cells (control mean=-49.77 ±6.57 mV, EN460 mean=-53.12 ±7.06 mV, F(1,297)=14.02, p=0.0002, d=0.045, 95% CI 1.59 to 5.11, Two-way ANOVA) and that this was true for both sexes (Male control mean=-50.0 ±6.73 mV, EN460 mean=-53.1 ±6.81 mV, p=0.0197. Female control mean=-49.5 ±6.44 mV, EN460 mean=-53.2 ±7.39 mV, p=0.0145, Sidak’s multiple comparisons test). Furthermore, we found that the current required to initiate an action potential, or rheobase, of EN460-treated cells was higher (control mean=18.78 ±17.60 pA, EN460 mean=80.63 ±85.32 pA, F(1, 186)=39.89, p<0.0001, d=0.18, 95% CI -81.17 to -42.53, Two-way ANOVA), and again this was true of both sexes (Male control mean=19.7 ± 19.92 pA, EN460 mean=106.3 ±101.65 pA, p<0.0001, Female control mean=17.8 ±15.30 pA, EN460 mean=54.9 ±49.70 pA, p=0.0167, Sidak’s multiple comparisons test) (FIG. 4D). These results are indicative of reduced excitability of DRG neurons after ERO1 inhibition.

Repetitive firing patterns in DRG neurons are highly linked to both acute and chronic pain.45,46 We observed that the action potential frequency of neurons was altered after ERO1 inhibition with EN460 (FIG. 4A, B). First, we noted that a smaller proportion of neurons appeared to be firing repetitively when we pooled the data from all current steps after ERO1 inhibition (FIG. 4F). The frequency of firing in response to 20 pA of current injection was significantly reduced in male neurons with ERO1 inhibition (control mean=4.92 ±5.32 Hz, EN460 mean=2.00 ±2.72 Hz, p=0.0484, student’s t-test) (FIG. 4G). In females, there was a significant reduction in action potential frequency in response to ERO1 inhibition at higher current injection steps (50 pA: control mean=9.65 ±5.28 Hz, EN460 mean=5.76 ±6.30 Hz p=0.032. 60 pA control mean=10.50 ±4.45 Hz, EN460 mean=6.75 ±7.29 Hz p=0.045, student’s t-test) (FIG. 4H). This reduction in action potential frequency suggests that ERO1 inhibition affects various aspects of DRG neuron excitability, which may be responsible for the dampened pain-like responses we have observed in vivo.

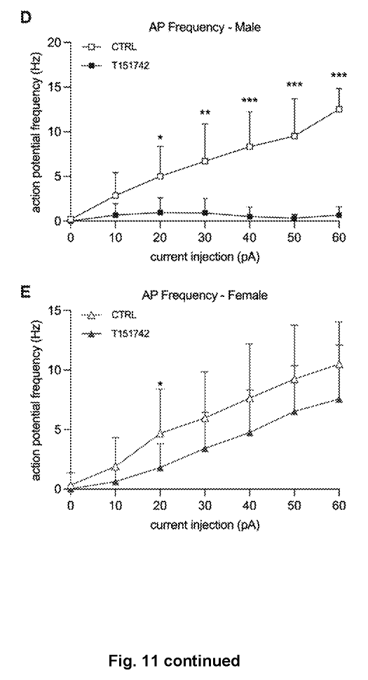

To confirm these results we used another inhibitor, T151742, as it has increased ERO1α specificity.³³ While EN460 is currently the standard for ERO1 inhibition and the only commercially available drug, it has some off-target effects on other enzymes. Using 5 μM T151742 produced a statistically significant decrease in the resting membrane potential (control mean=-47.16 ±4.83 mV, EN460 mean=-54.27 ±5.75, F(1,178)=61.45, p<0.0001, d=0.21, 95% CI 5.32 to 8.90, Two-way ANOVA). This was true individually of each sex (Male control mean=-46.9 ±6.42 mV, EN460 mean=-55.1 ±7.20 mV, p<0.0001. Female control mean=-47.4 ±4.24 mV, EN460 mean=-53.4 ±4.91 mV, p<0.0001, Sidak’s multiple comparisons test). The drug also increased rheobase (control mean=23.08 ±28.86 pA, EN460 mean=134.0 ±132.40 pA, F(1,116)=31.38, p<0.0001, d=0.26, 95% CI -150.2 to -71.72, Two-way ANOVA), which was true of both sexes (Male control mean=14.2 ±9.96 pA, EN460 mean=86.2 ±53.15 pA, p=0.034. Female control mean=32.0 ±31.39 pA, EN460 mean=181.9 ±152.75 pA, p<0.0001, Uncorrected Fisher’s LSD) (FIG. 11A, B). Consistent with the observed rheobase difference, there also appeared to be a reduction in the proportion of cells firing repetitively with T151742 treatment, like the effects we observed with EN460 ( FIG. 11C). Finally, there was a strong reduction in action potential frequency in male cells treated with T151742 from 20-60 pA of current injection (20 pA: control mean=5.00 ±3.36 Hz, EN460 mean=0.95 ±1.64 Hz p=0.014. 30 pA: control mean=6.71 ±4.16 Hz, EN460 mean=0.90 ±1.62 Hz p=0.0048. 40 pA control mean=8.33 ±3.92 Hz, EN460 mean=0.50 ±1.07 Hz p<0.001. 50 pA control mean=9.52 ±4.16 Hz, EN460 mean=0.33 ±0.41 Hz p<0.001. 60 pA control mean=12.53 ±2.29 Hz, EN460 mean=0.67 ±0.94 Hz p= p<0.001, student’s t-test). These effects were more modest in females however, with a significant reduction at 20 pA of current (control mean=4.68 ±3.73 Hz, EN460 mean=1.82 ±1.99 Hz, p=0.025, student’s t-test) (FIG. 11D/E). The cause of this sex difference is currently unclear, and further testing of the drug in vivo will be required to better understand its effects. Nonetheless, these results demonstrate that another ERO1 inhibitor can replicate similar effects to those seen with EN460.

### 4.5 ERO1 inhibition dampens mitochondrial function in mouse dorsal root ganglia in vitro.

We hypothesized that the reduced pain-like behaviour and DRG neuron excitability we observed with ERO1 inhibition may be due to effects on mitochondrial function caused by reduced calcium transfer at ERMCS. We investigated this in cultured DRGs using three readouts of mitochondrial function. Reduced calcium uptake by mitochondria would theoretically dampen mitochondrial bioenergetics, so we assessed mitochondrial function by measuring mitochondrial membrane potential (ΔΨm), ATP, and ROS production.^{12,13,47}

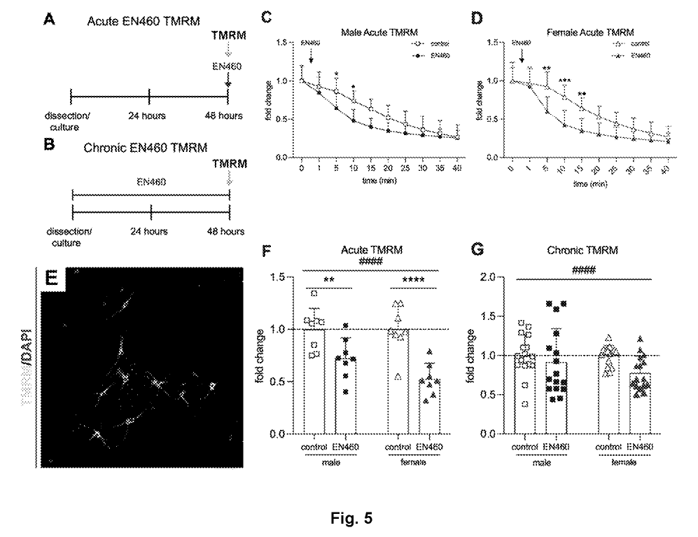

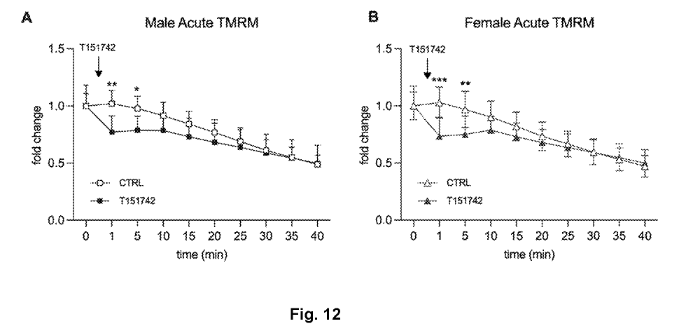

We first used TMRM to stain for ΔΨm after both acute and chronic ERO1 inhibition with EN460 in cultured DRG neurons (FIGS. 5A,E ). TMRM accumulates more and therefore is brighter in mitochondria with a more negatively charged matrix and therefore high ΔΨm. This signal dims if ΔΨm potential decreases.³⁴ For the acute paradigm we took a baseline measurement of TMRM stain intensity, then delivered EN460 and took another measurement immediately, followed by measurements in 5-minute intervals for 40 minutes. The acute timelapse curves (FIGS. 5C,D ) which are expressed as fold change of TMRM intensity over respective baselines show that male TMRM intensity was significantly reduced at 10 and 15 minutes following EN460 treatment (10 minutes: control mean=0.74 ±0.13, EN460 mean=0.48 ±0.15, p=0.010. 15 minutes: control mean=0.63 ±0.13, EN460 mean=0.40 ±0.11, p=0.030, Sidak’s multiple comparisons test, Repeated Measures Two-way ANOVA). Similarly, female TMRM intensity was significantly reduced 5, 10, and 15 minutes after EN460 treatment (5 minutes: control mean=0.92 ±0.19, EN460 mean=0.60 ±0.19, p=0.0015. 10 minutes: control mean=0.79 ±0.16, EN460 mean=0.43 ±0.19, p=0.0002. 15 minutes: control mean=0.64 ±0.14, EN460 mean=0.35 ±0.16, p=0.0045, Sidak’s multiple comparisons test, Repeated Measures Two-way ANOVA). When we expressed raw TMRM fluorescent intensity over time we found that the area under the curve was decreased during the first 40 minutes of EN460 treatment (FIG. 5F) (control mean=1.00 ±0.20, EN460 mean=0.62 ±0.20, F(1,28)=30.17, p<0.0001, d=0.52, 95% CI 0.24 to 0.52, Two-way ANOVA), and that this was true in both sexes (male control mean=1.00 ±0.20, EN460 mean=0.72 ±0.20, p=0.0079. Female control mean=1.00 ±0.22, EN460 mean=0.53 ±0.15, p<0.0001, Sidak’s multiple comparisons test). These results suggest that ERO1 inhibition with EN460 acutely reduces ΔΨm. Additionally, ERO1 inhibition with “chronic” (48 hours continuous) EN460 treatment (followed by a 30 minute treatment washout period during staining) also significantly lessened TMRM intensity (FIG. 5G) (control mean=1.00 ±0.21, EN460 mean=0.85 ±0.34, F(1,60)=4.767, p=0.0329, d=0.074, 95% CI 0.013 to 0.29, Two-way ANOVA), suggesting this effect can also be seen after our “chronic” treatment. However, on the level of individual sex this effect was only statistically in females (Male control mean=1.00 ±0.27, EN460 mean=0.92 ±0.43, p=0.40. Female control mean=1.00 ±0.13, EN460 mean=0.78 ±0.22, p=0.029, Uncorrected Fisher’s LSD test). The acute TMRM assay was also replicated using T151742 to confirm that ERO1 inhibition can affect ΔΨm, and we found that the ΔΨm was transiently reduced in both sexes with 5 μM T151742 (Female 1 minute: control mean=1.03 ±0.14, EN460 mean=0.73 ±0.17, p=0.0001, 5 minutes: control mean=0.97 ±0.16, EN460 mean=0.75 ±0.16, p=0.0090. Male 1 minute: control mean=1.02 ±0.11, EN460 mean=0.77 ±0.14, p=0.0032. Male 5 minutes: control mean=0.98 ±0.11, EN460 mean=0.79 ±0.12, p=0.049, Sidak’s multiple comparisons test, Repeated Measures Two-way ANOVA) (FIG. 12). Together, these results suggest that ERO1 inhibition can reduce ΔΨm, which may imply lessened metabolic function and ATP output, reducing ROS production as a byproduct. These changes may be responsible for the reduced sensory neuron excitability we observed in vitro and reduced nociceptive behaviour in vivo.

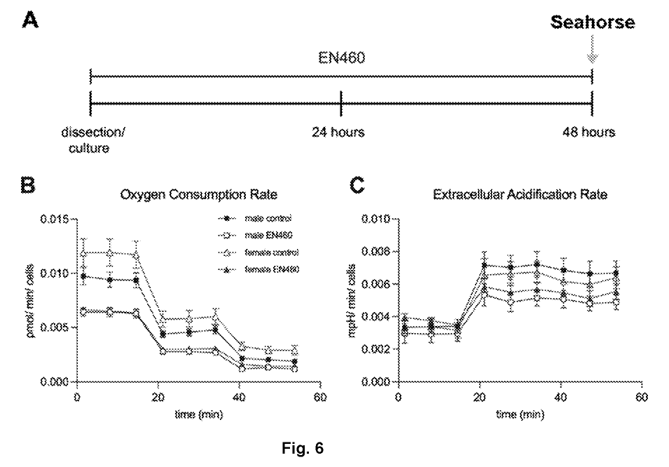

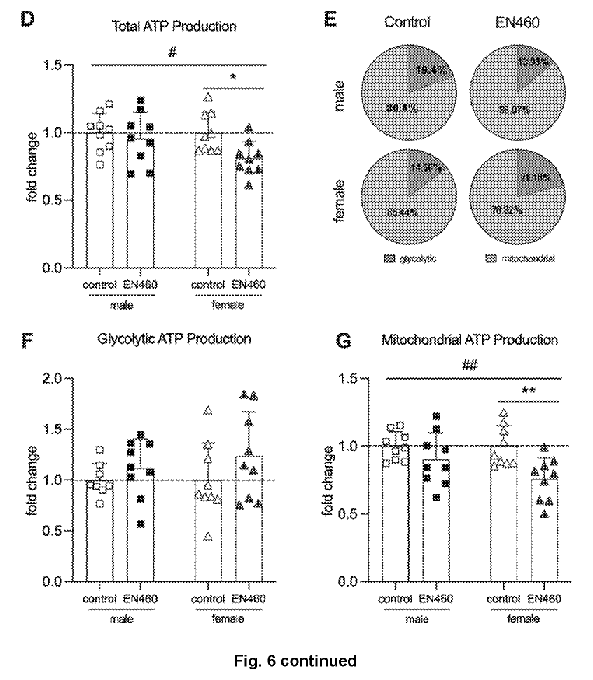

Next, we wanted to test the functional consequences of a lessened ΔΨm in response to ERO1 inhibition with EN460. We used an Agilent Seahorse Oxygraph to measure ATP production in our “chronic” paradigm (48 hours of continuous EN460 treatment followed by an hour of degassing without treatment) (FIG. 6A). The seahorse measures oxygen consumption as a proxy for mitochondrial ATP production (FIG. 6B), and extracellular acidification rate (ECAR) as a proxy for glycolytic ATP production (FIG. 6C). The representative traces shown are each from a single experiment out of three total. We saw that total ATP production was reduced (FIG. 6D) (control mean=1.00 ±0.14, EN460 mean=0.88 ±0.17, F(1,32)=5.142, p=0.0303, d=0.14, 95% CI 0.012 to 0.22, Two-way ANOVA) and that female cells in particular, exhibited decreased ATP production after EN460 treatment compared to untreated wells (Male control mean=1.00 ±0.14, EN460 mean=0.96 ±0.19, p=0.80. Female control mean=1.00 ±0.15, EN460 mean=0.81 ±0.13, p=0.0278, Sidak’s multiple comparisons test). When analysing the total ATP production in terms of glycolytic versus mitochondrial contributions (FIG. 6E-G) we saw no statistically significant changes in glycolytic ATP (control mean=1.00 ±0.28, EN460 mean=1.18 ±0.36, F(1,31)=2.50, p=0.12, d=0.0.075, 95% CI -0.41 to 0.051, Two-way ANOVA), but a significant reduction in mitochondrial ATP production with EN460 treatment (control mean=1.00 ±0.13, EN460 mean=0.83 ±0.19, F(1,32)=10.94, p=0.0023, d=0.25, 95% CI 0.066 to 0.28, Two-way ANOVA). Again, female cells were particularly affected by the drug (Male control mean=1.00 ±0.11, EN460 mean=0.90 ±0.20, p=0.34. Female control mean=1.00 ±0.15, EN460 mean=0.76 ±0.16, p=0.0043, Sidak’s multiple comparisons test). These results suggest that EN460 treatment reduces mitochondrial ATP production, and due to the use of the chronic paradigm, that these effects last at least two hours (accounting for degassing and total assay time) after treatment in vitro.

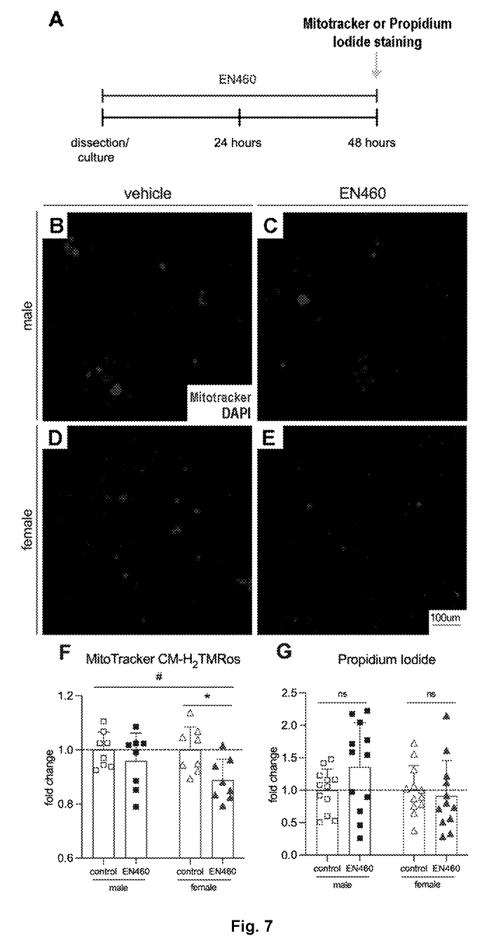

Mitochondrial ATP production via oxidative phosphorylation is a major source of mitochondrial ROS production. Based on our findings, we hypothesized that with the decreased mitochondrial ATP production in response to ERO1 inhibition would lead to an overall reduction in mitochondrial ROS production. To study this, we used Mitotracker CM-H2TMRos indictor which fluoresces in an oxidizing mitochondrial environment and indicates the presence of ROS. The cells were stained after chronic EN460 treatment then fixed before imaging (FIGS. 7A-F ). There was a significant reduction in the percentage of cells positive for the indicator, (control mean=1.00 ±0.073, EN460 mean=0.92 ±0.095, i(1,28)=6.65, p=0.015, d=0.19, 95% CI 0.016 5o 0.14, Two-way ANOVA). Like our TMRM findings, this effect was particularly strong in female neurons (Male control mean=1.00 ±0.066, EN460 mean=0.96 ±0.10, p=0.56. Female control mean=1.00 ±0.085, EN460 mean=0.89 ±0.078, p=0.025, Sidak’s multiple comparisons test). This indication that less cells were producing mitochondrial ROS agrees with our finding that cells were also producing less mitochondrial ATP.

Finally, we wanted to confirm that the dampened mitochondrial activity observed in the previous experiments was not linked to EN460-induced cell death. We stained EN460-treated cells for the cell death marker propidium iodide, an indicator that can only enter cells after death when membrane integrity is compromised.48 We did not find a significant change in the percentage of cells positive for this indicator (control mean=1.00 ±0.35, EN460 mean=1.14 ±0.64, F(1,44)=0.8872, p=0.3514, d=0.020, 95% CI -0.43 to 0.16, Two-way ANOVA. Male control mean=1.00 ±0.33, EN460 mean=1.36 ±0.68, p=0.1657. Female control mean=1.00 ±0.38, EN460 mean=0.91 ±0.54, p=0.8950, Sidak’s multiple comparisons test) (FIG. 7G). These results suggest that while the drug dampens mitochondrial function, these effects are not related to increased cell death.

### 4.6 ERO1α is expressed in post-mortem human dorsal root ganglia neurons.

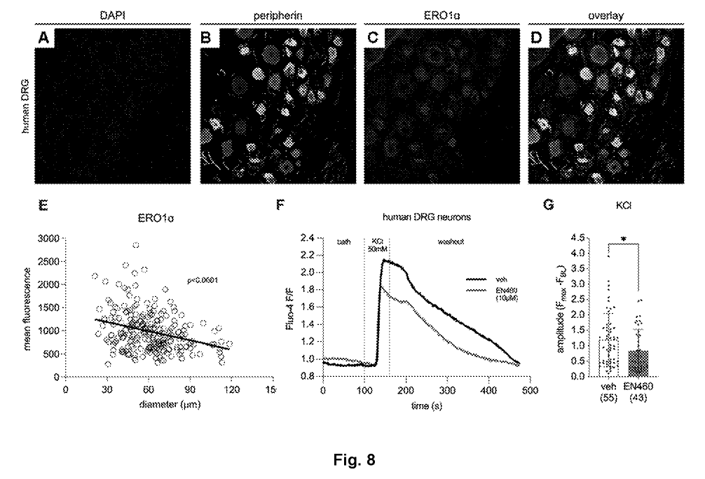

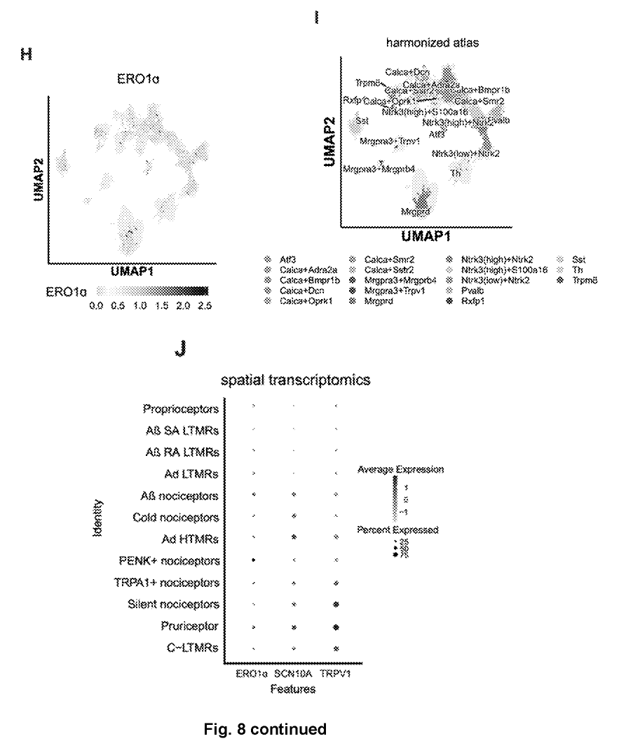

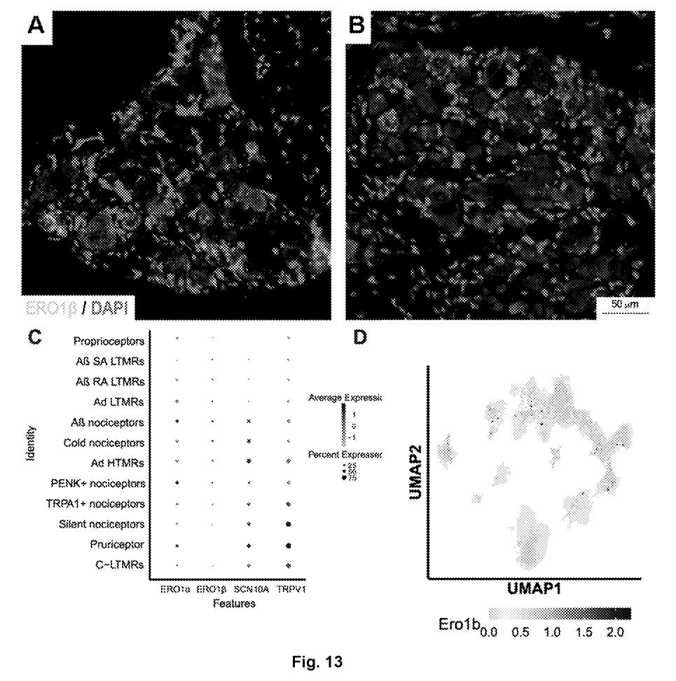

We found that ERO1α is expressed in three main mouse DRG neuron subtypes using immunohistochemistry. To validate ERO1 as a clinically relevant pain target, we wanted to confirm that it is expressed in human DRG neurons. Using immunohistochemistry, we found that ERO1α is expressed in human DRG neurons (FIG. 8A-D). We noticed that ERO1α fluorescence intensity was higher in smaller diameter neurons (FIG. 8E). Using a linear regression, we found that the slope was significantly non-zero and that the soma diameter was negatively correlated with ERO1α fluorescence intensity (y=-6.595x+1383, F(1, 226)=22.68, p<0.0001). Soma diameter can be used to identify neuronal subtypes, and human nociceptors tend to have a diameter <60 μm.^{49,50} This suggests that ERO1α may be more highly expressed in human nociceptors than other sensory neuron subtypes. To further investigate ERO1α expression in human DRG neuron subtypes we used previously published single-cell and spatial RNA sequencing dataset.⁵¹ This data shows that ERO1α mRNA is expressed across human DRG neuron subtypes (FIG. 8H-J). We also stained mouse DRGs and investigated the human DRG RNA sequencing data for expression of the β isoform of ERO1 (FIG. 13). While we did see some expression, it appeared to be a lesser amount in a lower percentage of cells than ERO1α. Together, these results show that ERO1α is expressed in human DRG neurons, making it a potential target for clinical pain therapeutics.

### 4.7 ERO1 inhibition reduces the sensitization of post-mortem human dorsal root ganglia neurons in vitro.

Finally, we assessed whether modulating ERO1 using EN460 impacts the calcium transients of human DRG neurons (FIG. 8F, G). Cultured human DRG neurons were treated with either a vehicle (0.04% DMSO) or with 10 μM EN460 for 30 min before being depolarized using 50 mM KCl. The peak amplitude of fluorescence was decreased in EN460-treated cells compared to vehicle-treated control cells (control mean=1.18 ±0.86, EN460 mean=0.84 ±0.69, unpaired t-test t(96)=2.113, p=0.0372, d=0.44). These results show that like our findings in mouse DRGs, ERO1 inhibition reduces human DRG neuron activity, suggesting that ERO1 inhibition may be a viable strategy for treating pain in humans.

### 5. Discussion

ERO1 has classically been studied as a target in cancer and skeletal muscle,^{52,53} but had not yet been investigated in peripheral sensory tissues. We began by staining for the broadly expressed subtype, ERO1α.^{38,39} In naïve mouse DRG tissue from both sexes we found that roughly 80-90% of neurons across subtypes express ERO1α. This was an important first step to suggest that ERO1 inhibition might affect sensory function. We then chose to begin our tests with the ERO1 inhibitor, EN460, which binds the active form of ERO1 to inhibit its activity. ERO1 promotes the open conformation of IP3Rs, a critical mechanism for calcium release from the ER, via its production of ROS. It also increases IP3R activity and increases its localization to ERMCS during cell stress.²⁵ We hypothesized that in painful conditions this may cause mitochondrial-linked IP3Rs to remain open, promoting high levels of calcium transfer. Therefore, inhibition of ERO1 should reduce calcium transfer, affecting mitochondrial function, which is associated with neuronal excitability.^{11,18} At a molecular weight of 460.79, EN460 is above the size that would be expected to cross the blood brain barrier,⁵⁴ therefore we elected to deliver it via intraperitoneal injection to restrict its activity to the periphery.

We began with an acute naïve model, the thermal tail flick test, where we showed that ERO1 inhibition with EN460 can modulate nociceptive withdrawal threshold with an ED50 of 15 mg/kg. Next, we tested inflammatory model, the formalin test, which evokes nocifensive behaviours such as shaking, licking, and guarding of the formalin injected paw. The formalin test also produces two classic phases of pain-like behaviour, the first phase is thought to represent acute sharp pain that follows an injury, and after a brief reprieve the second phase represents a dull aching allodynic pain due to inflammation.⁵⁵ ERO1 inhibition with EN460 at a low dose significantly reduced the first phase of behaviours, and fully abolished the second phase. At a high dose both phases of behaviour were abolished. We repeated this experiment with a naloxone pre-treatment to assess whether these effects were mediated by an endogenous opioid mechanism. However, pre-treatment with naloxone had no discernable impact on the analgesia produced by EN460. We also performed a rotarod test and verified that the reduction in pain-like behaviours that we observed with EN460 was not due to impaired motor function. After assessing nociceptive behaviours, we collected tissue from these animals and stained for a neuronal activation marker, c-FOS, in the dorsal horn of the spinal cord where the afferents of primary sensory neurons synapse. We found a high proportion of c-FOS-stained cells in the animals treated with formalin alone, but there was a significant reduction in c-FOS expression in formalin animals treated with EN460, suggesting reduced afferent drive into the spinal cord with EN460 treatment.

We also investigated a more clinically relevant model, the post-incisional model of post-surgical pain. This model involves an incision into the mouse hind paw skin and muscle under general anesthetic. We then treated mice daily dose with EN460 and measured thermal and mechanical withdrawal thresholds 30 minutes after drug treatment. With this paradigm we were able to determine that EN460 transiently reduced mechanical post-surgical allodynia. However, there was no statistically significant effect on thermal hyperalgesia, which might indicate a difference in ERO1 expression and/ or function in the nociceptor sub-population that senses noxious heat.

Using patch clamp electrophysiology in our DRG cultures, we saw multiple changes with EN460 treatment. The resting membrane potential of the treated neurons was lower, and the rheobase was higher, indicating more current was required to initiate an action potential. This suggests that ERO1 inhibition could make sensory neurons harder to stimulate. Additionally, the proportion of repetitively firing cells and the frequency of action potentials from these cells was lower with EN460 treatment. As high frequency nociceptor firing is a hallmark feature of pain,^{45,46} this effect of ERO1 inhibition may also contribute to the reduced pain-like behaviours we observed in mice. However, EN460 acts on ERO1 via flavin adenosine dinucleotide (FAD) binding, and for this reason it can also inhibit monoamine oxidases (MAOs).⁵⁶ Because MAO inhibitors may influence mitochondrial function,^{57,58} we wanted to confirm our observations with another inhibitor of ERO1α. To verify our results we also used the sulfuretin derivative, T151742, which has greater ERO1α specificity and less potent effects on other FAD-containing enzymes.³³ With this drug we were able to replicate a similar profile of electrophysiological effects indicating that ERO1 inhibition is responsible for reduced sensory neuron excitability.

We also used our culture model to investigate readouts of mitochondrial function to support our hypothesis that ERO1 inhibition reduces neuronal excitability and nociception through a reduction in ER-mitochondrial calcium transfer. We hypothesized that if this were true, we would see a dampening of mitochondrial function with ERO1 inhibition, and we tested this by assessing ΔΨm, ATP, and ROS production. All three of these measurements were decreased with EN460 treatment compared to untreated wells, suggesting that ERO1 inhibition with EN460 influences ERMCS calcium transfer and mitochondrial function. We also tested T151742 in our acute TMRM assay and found the effect to be slightly less dramatic, and more transient, but still present. This supports our prediction that ERO1 inhibition would influence mitochondrial activity.

Finally, we replicated some of our results in post-mortem human DRG tissue. We confirmed via immunohistochemistry and analysis of single cell RNA sequencing data that ERO1α is expressed in human sensory neurons across subtypes. Immunostaining also revealed that ERO1α expression is higher in small diameter afferents, which are the population mainly attributed to nociceptors. These results may indicate that ERO1 inhibition could have greater effects on nociception than non-noxious sensation. Finally, we used calcium imaging to assess human DRG neuron sensitization in vitro and found it to be reduced in response to EN460.

While we found a clear effect of ERO1 inhibition on mouse DRG neuron mitochondrial function, excitability and pain-like behaviour, this work is not without limitations. We cannot prove definitively that dampened mitochondrial function is the only mechanism responsible for reduced excitability and pain behaviour. While we predict ERMCS calcium transfer to be a major contributing mechanism due to the localization of ERO1 to ERMCS,²⁵ our results do not rule out possible contributions of changes to cytosolic calcium, or changes in protein folding. Due to the nature of adult DRG cultures, we cannot exclude a possible contribution of non-neuronal cells to altered neuronal sensitivity and nociception. We also cannot exclude the possibility that inhibition of ERO1 in other tissues is contributing to the behavioural effects demonstrated here.

In summary, our results show that ERO1 is expressed in both mouse and human DRG neurons, and its inhibition with EN460 reduces the activity of these neurons in both species. This translates to reduced acute inflammatory pain-like behaviours and mechanical post-surgical pain-like behaviours in mice. We hypothesize that these results are due to reduced calcium transfer at ERMCS and subsequently dampened mitochondrial function, which we see with ERO1 inhibition in mouse DRGs in vitro. We conclude that ERO1 inhibition, and more broadly ER-mitochondrial calcium transfer, are a promising therapeutic avenue for non-narcotic acute pain relief, and we are optimistic that further investigation may even reveal this pathway as a target in chronic pain.

### REFERENCES

1. Yousuf MS, Maguire AD, Simmen T, Kerr BJ: Endoplasmic reticulum-mitochondria interplay in chronic pain: The calcium connection. Mol Pain 2020; 16: 1744806920946889

2. Bourinet E, Altier C, Hildebrand ME, Trang T, Salter MW, Zamponi GW: Calcium-permeable ion channels in pain signaling. Physiol Rev 2014; 94: 81-140

3. Verkhratsky A: Physiology and pathophysiology of the calcium store in the endoplasmic reticulum of neurons. Physiol Rev 2005; 85: 201-79

4. Taylor AMW: Peripheral mechanisms of trigeminal neuropathic pain, Department of Pharmacology and Therapeutics. McGill University, 2011

5. Park J, Luo ZD: Calcium channel functions in pain processing. Channels (Austin) 2010; 4: 510-7

6. Veeresh P, Kaur H, Sarmah D, Mounica L, Verma G, Kotian V, Kesharwani R, Kalia K, Borah A, Wang X, Dave KR, Rodriguez AM, Yavagal DR, Bhattacharya P: Endoplasmic reticulum-mitochondria crosstalk: from junction to function across neurological disorders. Ann N Y Acad Sci 2019; 1457: 41-60

7. Vance JE: MAM (mitochondria-associated membranes) in mammalian cells: lipids and beyond. Biochim Biophys Acta 2014; 1841: 595-609

8. Krebs J, Agellon LB, Michalak M: Ca(2+) homeostasis and endoplasmic reticulum (ER) stress: An integrated view of calcium signaling. Biochem Biophys Res Commun 2015; 460: 114-21

9. Filadi R, Theurey P, Pizzo P: The endoplasmic reticulum-mitochondria coupling in health and disease: Molecules, functions and significance. Cell Calcium 2017; 62: 1-15

10. Marchi S, Pinton P: The mitochondrial calcium uniporter complex: molecular components, structure and physiopathological implications. J Physiol 2014; 592: 829-39

11. Flatters SJ: The contribution of mitochondria to sensory processing and pain. Prog Mol Biol Transl Sci 2015; 131: 119-46

12. Lee SH, Duron HE, Chaudhuri D: Beyond the TCA cycle: new insights into mitochondrial calcium regulation of oxidative phosphorylation. Biochem Soc Trans 2023; 51: 1661-1673

13. Wescott AP, Kao JPY, Lederer WJ, Boyman L: Voltage-energized Calcium-sensitive ATP Production by Mitochondria. Nat Metab 2019; 1: 975-984

14. Chaban VV: Peripheral sensitization of sensory neurons. Ethn Dis 2010; 20: S1-3-6

15. Yousuf MS, Samtleben S, Lamothe SM, Friedman TN, Catuneanu A, Thorburn K, Desai M, Tenorio G, Schenk GJ, Ballanyi K, Kurata HT, Simmen T, Kerr BJ: Endoplasmic reticulum stress in the dorsal root ganglia regulates large-conductance potassium channels and contributes to pain in a model of multiple sclerosis. FASEB J 2020; 34: 12577-12598

16. Salvemini D, Little JW, Doyle T, Neumann WL: Roles of reactive oxygen and nitrogen species in pain. Free Radic Biol Med 2011; 51: 951-66

17. Ohtake Y, Matsuhisa K, Kaneko M, Kanemoto S, Asada R, Imaizumi K, Saito A: Axonal Activation of the Unfolded Protein Response Promotes Axonal Regeneration Following Peripheral Nerve Injury. Neuroscience 2018; 375: 34-48

18. Medvedeva YV, Kim MS, Usachev YM: Mechanisms of prolonged presynaptic Ca2+ signaling and glutamate release induced by TRPV1 activation in rat sensory neurons. J Neurosci 2008; 28: 5295-311

19. Kidd JF, Pilkington MF, Schell MJ, Fogarty KE, Skepper JN, Taylor CW, Thorn P: Paclitaxel affects cytosolic calcium signals by opening the mitochondrial permeability transition pore. J Biol Chem 2002; 277: 6504-10

20. Kostyuk E, Svichar N, Shishkin V, Kostyuk P: Role of mitochondrial dysfunction in calcium signalling alterations in dorsal root ganglion neurons of mice with experimentally-induced diabetes. Neuroscience 1999; 90: 535-41

21. George DS, Hackelberg S, Jayaraj ND, Ren D, Edassery SL, Rathwell CA, Miller RE, Malfait AM, Savas JN, Miller RJ, Menichella DM: Mitochondrial calcium uniporter deletion prevents painful diabetic neuropathy by restoring mitochondrial morphology and dynamics. Pain 2022; 163: 560-578

22. Ramming T, Kanemura S, Okumura M, Inaba K, Appenzeller-Herzog C: Cysteines 208 and 241 in Ero1alpha are required for maximal catalytic turnover. Redox Biol 2016; 7: 14-20

23. Li G, Mongillo M, Chin KT, Harding H, Ron D, Marks AR, Tabas I: Role of ERO1-alpha-mediated stimulation of inositol 1,4,5-triphosphate receptor activity in endoplasmic reticulum stress-induced apoptosis. J Cell Biol 2009; 186: 783-92

24. Gilady SY, Bui M, Lynes EM, Benson MD, Watts R, Vance JE, Simmen T: Ero1alpha requires oxidizing and normoxic conditions to localize to the mitochondria-associated membrane (MAM). Cell Stress Chaperones 2010; 15: 619-29

25. Bassot A, Chen J, Takahashi-Yamashiro K, Yap MC, Gibhardt CS, Le GNT, Hario S, Nasu Y, Moore J, Gutierrez T, Mina L, Mast H, Moses A, Bhat R, Ballanyi K, Lemieux H, Sitia R, Zito E, Bogeski I, Campbell RE, Simmen T: The endoplasmic reticulum kinase PERK interacts with the oxidoreductase ERO1 to metabolically adapt mitochondria. Cell Rep 2023; 42: 111899

26. Olechowski CJ, Parmar A, Miller B, Stephan J, Tenorio G, Tran K, Leighton J, Kerr BJ: A diminished response to formalin stimulation reveals a role for the glutamate transporters in the altered pain sensitivity of mice with experimental autoimmune encephalomyelitis (EAE). Pain 2010; 149: 565-572

27. Lara Aparicio SY, Laureani Fierro AJ, Aranda Abreu GE, Toledo Cardenas R, Garcia Hernandez LI, Coria Avila GA, Rojas Duran F, Aguilar MEH, Manzo Denes J, Chi-Castaneda LD, Perez Estudillo CA: Current Opinion on the Use of c-Fos in Neuroscience. NeuroSci 2022; 3: 687-702

28. Friedman TN, La Caprara O, Zhang C, Lee K, May J, Faig CA, Baldwin T, Plemel JR, Taylor AMW, Kerr BJ: Sex differences in peripheral immune cell activation: Implications for pain and pain resolution. Brain Behav Immun 2023; 114: 80-93

29. Chaplan SR, Bach FW, Pogrel JW, Chung JM, Yaksh TL: Quantitative assessment of tactile allodynia in the rat paw. J Neurosci Methods 1994; 53: 55-63

30. Dixon WJ: Efficient analysis of experimental observations. Annu Rev Pharmacol Toxicol 1980; 20: 441-62

31. Cheah M, Fawcett JW, Andrews MR: Assessment of Thermal Pain Sensation in Rats and Mice Using the Hargreaves Test. Bio Protoc 2017; 7

32. Maguire AD, Plemel JR, Kerr BJ: Enrichment of Adult Mouse Dorsal Root Ganglia Neuron Cultures by Immunopanning. J Vis Exp 2023

33. Johnson BD, Kaulagari S, Chen WC, Hayes K, Geldenhuys WJ, Hazlehurst LA: Identification of Natural Product Sulfuretin Derivatives as Inhibitors for the Endoplasmic Reticulum Redox Protein ERO1alpha. ACS Bio Med Chem Au 2022; 2: 161-170

34. Joshi DC, Bakowska JC: Determination of mitochondrial membrane potential and reactive oxygen species in live rat cortical neurons. J Vis Exp 2011

35. David Y, Mei, Jain, Krishnagiri, Srikanth, Dussor, Dalva, Price: Ephrin-B2 promotes nociceptive plasticity and hyperalgesic priming through EphB2-MNK-eIF4E signaling in both mice and humans. bioRxiv 2024

36. Yousuf MS, Sahn JJ, Yang H, David ET, Shiers S, Mancilla Moreno M, Iketem J, Royer DM, Garcia CD, Zhang J, Hong VM, Mian SM, Ahmad A, Kolber BJ, Liebl DJ, Martin SF, Price TJ: Highly specific sigma(2)R/TMEM97 ligand FEM-1689 alleviates neuropathic pain and inhibits the integrated stress response. Proc Natl Acad Sci U S A 2023; 120: e2306090120

37. Valtcheva MV, Copits BA, Davidson S, Sheahan TD, Pullen MY, McCall JG, Dikranian K, Gereau RWt: Surgical extraction of human dorsal root ganglia from organ donors and preparation of primary sensory neuron cultures. Nat Protoc 2016; 11: 1877-88

38. Shergalis AG, Hu S, Bankhead A, 3rd, Neamati N: Role of the ERO1-PDI interaction in oxidative protein folding and disease. Pharmacol Ther 2020; 210: 107525

39. Dias-Gunasekara S, Gubbens J, van Lith M, Dunne C, Williams JA, Kataky R, Scoones D, Lapthorn A, Bulleid NJ, Benham AM: Tissue-specific expression and dimerization of the endoplasmic reticulum oxidoreductase Ero1beta. J Biol Chem 2005; 280: 33066-75

40. Ernfors ECEaP: Dorsal Root Ganglion Neuron Types and Their Functional Specialization. The Oxford Handbook of the Neurobiology of Pain 2018

41. Ishikawa T, Miyagi M, Ohtori S, Aoki Y, Ozawa T, Doya H, Saito T, Moriya H, Takahashi K: Characteristics of sensory DRG neurons innervating the lumbar facet joints in rats. Eur Spine J 2005; 14: 559-64

42. Perry MJ, Lawson SN, Robertson J: Neurofilament immunoreactivity in populations of rat primary afferent neurons: a quantitative study of phosphorylated and non-phosphorylated subunits. J Neurocytol 1991; 20: 746-58

43. Cowie AM, Stucky CL: A Mouse Model of Postoperative Pain. Bio Protoc 2019; 9

44. Ulker E, Caillaud M, Koseli E, Contreras K, Alkhlaif Y, Lindley E, Barik M, Ghani S, Bryant CD, Imad Damaj M: Comparison of Pain-Like behaviors in two surgical incision animal models in C57BL/6J mice. Neurobiol Pain 2022; 12: 100103

45. Jagodic MM, Pathirathna S, Nelson MT, Mancuso S, Joksovic PM, Rosenberg ER, Bayliss DA, Jevtovic-Todorovic V, Todorovic SM: Cell-specific alterations of T-type calcium current in painful diabetic neuropathy enhance excitability of sensory neurons. J Neurosci 2007; 27: 3305-16

46. von Hehn CA, Baron R, Woolf CJ: Deconstructing the neuropathic pain phenotype to reveal neural mechanisms. Neuron 2012; 73: 638-52

47. Finkel T, Menazza S, Holmstrom KM, Parks RJ, Liu J, Sun J, Liu J, Pan X, Murphy E: The ins and outs of mitochondrial calcium. Circ Res 2015; 116: 1810-9

48. Crowley LC, Scott AP, Marfell BJ, Boughaba JA, Chojnowski G, Waterhouse NJ: Measuring Cell Death by Propidium Iodide Uptake and Flow Cytometry. Cold Spring Harb Protoc 2016; 2016

49. Shiers S, Klein RM, Price TJ: Quantitative differences in neuronal subpopulations between mouse and human dorsal root ganglia demonstrated with RNAscope in situ hybridization. Pain 2020; 161: 2410-2424

50. Tavares-Ferreira D, Shiers S, Ray PR, Wangzhou A, Jeevakumar V, Sankaranarayanan I, Cervantes AM, Reese JC, Chamessian A, Copits BA, Dougherty PM, Gereau RWt, Burton MD, Dussor G, Price TJ: Spatial transcriptomics of dorsal root ganglia identifies molecular signatures of human nociceptors. Sci Transl Med 2022; 14: eabj8186

51. Bhuiyan: Harmonized cross-species cell atlases of trigeminal and dorsal root ganglia. bioRxiv 2023

52. Varone E, Chernorudskiy A, Cherubini A, Cattaneo A, Bachi A, Fumagalli S, Erol G, Gobbi M, Lenardo MJ, Borgese N, Zito E: ERO1 alpha deficiency impairs angiogenesis by increasing N-glycosylation of a proangiogenic VEGFA. Redox Biol 2022; 56: 102455

53. Germani S, Van Ho AT, Cherubini A, Varone E, Chernorudskiy A, Renna GM, Fumagalli S, Gobbi M, Lucchetti J, Bolis M, Guarrera L, Craparotta I, Rastelli G, Piccoli G, de Napoli C, Nogara L, Poggio E, Brini M, Cattaneo A, Bachi A, Simmen T, Cali T, Quijano-Roy S, Boncompagni S, Blaauw B, Ferreiro A, Zito E: SEPN1-related myopathy depends on the oxidoreductase ERO1A and is druggable with the chemical chaperone TUDCA. Cell Rep Med 2024; 5: 101439

54. Kadry H, Noorani B, Cucullo L: A blood-brain barrier overview on structure, function, impairment, and biomarkers of integrity. Fluids Barriers CNS 2020; 17: 69

55. Abbott FV, Franklin KBJ, Westbrook FR: The formalin test: scoring properties of the first and second phases of the pain response in rats. Pain 1995; 60: 91-102

56. Hayes KE, Batsomboon P, Chen WC, Johnson BD, Becker A, Eschrich S, Yang Y, Robart AR, Dudley GB, Geldenhuys WJ, Hazlehurst LA: Inhibition of the FAD containing ER oxidoreductin 1 (Ero1) protein by EN-460 as a strategy for treatment of multiple myeloma. Bioorg Med Chem 2019; 27: 1479-1488

57. Sorato E, Menazza S, Zulian A, Sabatelli P, Gualandi F, Merlini L, Bonaldo P, Canton M, Bernardi P, Di Lisa F: Monoamine oxidase inhibition prevents mitochondrial dysfunction and apoptosis in myoblasts from patients with collagen VI myopathies. Free Radic Biol Med 2014; 75: 40-7

58. Kaludercic N, Carpi A, Nagayama T, Sivakumaran V, Zhu G, Lai EW, Bedja D, De Mario A, Chen K, Gabrielson KL, Lindsey ML, Pacak K, Takimoto E, Shih JC, Kass DA, Di Lisa F, Paolocci N: Monoamine oxidase B prompts mitochondrial and cardiac dysfunction in pressure overloaded hearts. Antioxid Redox Signal 2014; 20: 267-80

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A method of treating pain in a subject in need thereof, comprising: administering a therapeutically effected amount an inhibitor of Endoplasmic Reticulum Oxidoreductin 1 (EOR1).

2. The method of claim 1, wherein the inhibitor of EOR1 comprises EN460 and/or T151742.

3. The method of claim 2, wherein the pain is acute pain, chronic pain, inflammatory pain, nociceptive pain, neuropathic pain, hyperalgesia, allodynia, central pain, cancer-related pain, post-operative pain, visceral pain, musculo-skeletal pain, heart or vascular pain, head pain including migraine, orofacial pain including dental pain, or back pain.

4. The method of claim 2, wherein the pain is nociceptive pain.

5. The method of claim 4, wherein the nociceptive pain is post-operative pain, cluster headache pain, dental pain, surgical pain, pain resulting from burns, pain resulting from sunburns, pain resulting from exposure to extremely cold temperatures, bruises, fractures, post-partum pain, angina pain, genitourinary tract related pain, and/or damage by contact with toxic of hazardous chemicals.

6. The method of claim 2, wherein the pain is inflammatory pain.

7. The method of claim 6, wherein the inflammatory pain is arthritic pain, pain resulting from osteoarthritis, pain resulting from rheumatoid arthritis, pain resulting from inflammatory bowel diseases, pain resulting from psoriasis, or pain resulting from eczema.

8. The method of claim 2, wherein the subject is a human.

9. A method of treating pain in a subject in need thereof, comprising:
assessing the subject to determine if they are experiencing pain, and if such pain is diagnosed;
administering to the patient a therapeutically effective amount of an inhibitor of Endoplasmic Reticulum Oxidoreductin 1 (EOR1).

10. The method of claim 9, wherein the inhibitor of EOR1 comprises EN460 and/or T151742.

11. The method of claim 10, wherein the pain is acute pain, chronic pain, inflammatory pain, nociceptive pain, neuropathic pain, hyperalgesia, allodynia, central pain, cancer-related pain, post-operative pain, visceral pain, musculo-skeletal pain, heart or vascular pain, head pain including migraine, orofacial pain including dental pain, or back pain.

12. The method of claim 10, wherein the pain is nociceptive pain.

13. The method of claim 12, wherein the nociceptive pain is post-operative pain, cluster headache pain, dental pain, surgical pain, pain resulting from burns, pain resulting from sunburns, pain resulting from exposure to extremely cold temperatures, bruises, fractures, post-partum pain, angina pain, genitourinary tract related pain, and/or damage by contact with toxic of hazardous chemicals.

14. The method of claim 10, wherein the pain is inflammatory pain.

15. The method of claim 14, wherein the inflammatory pain is arthritic pain, pain resulting from osteoarthritis, pain resulting from rheumatoid arthritis, pain resulting from inflammatory bowel diseases, pain resulting from psoriasis, or pain resulting from eczema.

16. The method of claim 10, wherein the subject is a human.
